(19) United States
(12) Patent Application Publication
(10) Pub. No.: US 2006/0160121 A1
(43) Pub. Date: Jul. 20, 2006

(54) Probe arrays for detecting multiple strains of different species

(75) Inventors: William Martin Mounts, Andover, MA (US); Ellen Murphy, City Island, NY (US); Stephen Bruce Olmsted, West Nyack, NY (US)

Correspondence Address: NIXON PEABODY LP, 401 9TH STREET, N.W., SUITE 900, WASHINGTON, DC, 20004, US

(73) Assignee: Wyeth, Madison, NJ (US)

(21) Appl. No.: 11/348,413

(22) Filed: Feb. 7, 2006

Related U.S. Application Data
(63) Continuation-in-part of application No. 11/243,445, filed on Oct. 5, 2005.
Application No. 11/243,445 is a continuation-in-part of application No. PCT/US05/35471, filed on Oct. 5, 2005.
(60) Provisional application No. 60/615,573, filed on Oct. 5, 2004.

(51) Int. Cl. C12Q 1/68; C12M 1/34

(52) U.S. Cl. 435/6; 435/287.2

(57) ABSTRACT

The present invention provides probe arrays and methods of using the same for concurrent and discriminable detection of multiple strains of different species. In one aspect, the probe arrays of the present invention are nucleic acid arrays comprising (1) a first group of probes, each of which is specific to a different respective strain of a first species; and (2) a second group of probes, each of which is specific to a different respective strain of a second species. In many embodiments, the nucleic acid arrays of the present invention further include a third group of probes, each of which is specific to a different strain of a third species. In one example, a nucleic acid array of the present invention includes probes for sequences selected from SEQ ID NOs: 1 to 18,598, and can discriminably detect different strains of Streptococcus pyogenes, Streptococcus agalactiae and Staphylococcus epidermidis.

This application is a continuation-in-part of U.S. patent application Ser. No. 11/243,445, filed Oct. 5, 2005, now pending, and International Application No. PCT/US05/035471, filed Oct. 5, 2005, now pending, both of which claim the benefit of U.S. Provisional Application No. 60/615,573, filed Oct. 5, 2004. All of these applications are incorporated herein by reference in their entireties.

This application incorporates by reference all materials on the compact discs labeled “Copy 1” and “Copy 2.” Each of the compact discs includes Sequence Listing.ST25.txt (394,168 KB, created on Jan. 31, 2006).

### TECHNICAL FIELD

This invention relates to probe arrays and methods of using the same for concurrent and discriminable detection of multiple strains of different species.

### BACKGROUND

Streptococcus pyogenes (Group A streptococcus) is one of the most frequent pathogens of humans and can cause a wide range of illnesses from noninvasive disease such as pharyngitis and pyoderma to more severe invasive infections (e.g., bacteremia, pneumonia and puerperal sepsis). Streptococcus pyogenes also contains antigens similar to those of human cardiac, skeletal, smooth muscle and neuronal tissues, leading to autoimmune reactions following some infections. Streptococcus pyogenes is susceptible to penicillin, which remains the drug of choice for treating infections by this organism. Erythromycin and other macrolides have been recommended as alternative treatments for patients allergic to penicillin; however, resistance to erythromycin and related drugs has been observed in certain Streptococcus pyogenes strains.

Streptococcus agalactiae (Group B streptococcus) has been reported with increasing frequency as the cause of a variety of human infections, such as pharyngitis, cellulitis, meningitis, endocarditis and sepsis. Almost half of the cases of invasive Streptococcus agalactiae disease occurs in newborns. Disease in infants usually occurs as bacteremia, pneumonia, or meningitis. Other syndromes (e.g., cellulitis and osteomyelitis) can also occur. Approximately 25% of the cases of neonatal Streptococcus agalactiae disease occurs in premature infants. In pregnant women, Streptococcus agalactiae infection causes urinary tract infection, amnionitis, endometritis, and wound infection; stillbirths and premature delivery also have been attributed to Streptococcus agalactiae infection. In addition, Streptococcus agalactiae has been recognized as a significant pathogen in adults, especially among patients with underlying conditions. Skin or soft tissue infection, bacteremia, genitourinary infection, and pneumonia are the common manifestations of Streptococcus agalactiae disease in nonpregnant adults. CDC active surveillance shows that over the past three years the case-fatality rate for Streptococcus agalactiae disease has remained fairly constant at about 10% across all age groups.

Penicillin and ampicillin are the drugs of choice for prevention and treatment of Streptococcus agalactiae infections, and clindamycin and erythromycin are the alternatives for patients who are allergic to β-lactam agents. Infections with penicillin-tolerant Streptococcus agalactiae have been described. Isolates resistant to erythromycin and clindamycin also have been reported.

Staphylococcus epidermidis is a gram-positive bacteria present in the normal flora of humans, and is typically present on the skin. Most strains of Staphylococcus epidermidis are nonpathogenic and may even play a protective role in their host as normal flora. However, some Staphylococcus epidermidis strains have been implicated in various human conditions and diseases, including subacute bacterial endocarditis and septicemia. Staphylococcus epidermidis is estimated to be responsible for about 12% of all hospital patient infections. Because of the organism's peculiar ability to colonize polymer and metallic surfaces, there is a correlation of infection with the insertion of intravenous lines or catheters or implantation of prosthetic devices. Treatment can be difficult since different isolates of Staphylococcus epidermidis show a broad spectrum of antibiotic resistance. In addition, Staphylococcus epidermidis can produce a polysaccharide biofilm which helps to protect the bacteria from the human immune system. The ability to form a biofilm on the surface of a prosthetic device is also believed to be a significant determinant of virulence for this bacterium.

The ability to promptly identify and classify different pathogens is often pivotal to the diagnosis, prophylaxis, or treatment of infectious disease. For instance, many methods that enable the identification of Staphylococcus aureus strains fail in the identification of Staphylococcus epidermidis or other coagulase-negative staphylococci. Atypical characteristics in certain Staphylococcus epidermidis strains also result in their misidentification as Staphylococcus hominis. Moreover, traditional detection methods such as 16S DNA analyses, serotyping or ribotyping are laborious, and many of these methods are incapable of discriminably detecting multiple strains of different pathogenic species at the same time. Therefore, there is a need for new methods that would allow rapid, accurate and discriminable detection of infectious pathogens.

### SUMMARY OF THE INVENTION

The present invention provides probe arrays that allow for concurrent and discriminable detection of multiple strains of different viral or non-viral species. In one aspect, the probe arrays of the present invention are nucleic acid arrays which comprise:

a first group of polynucleotide probes, each of which is specific to a different respective strain of a first species; and

a second group of polynucleotide probes, each of which is specific to a different respective strain of a second species.

The nucleic acid arrays of the present invention can also comprise a third group of polynucleotide probes, each of which is specific to a different respective strain of a third species.

Non-viral species amenable to the present invention include, but are not limited to, β-hemolytic streptococci (e.g., Streptococcus pyogenes or Streptococcus agalactiae), Staphylococcus spp. (e.g., Staphylococcus epidermidis or Staphylococcus aureus), or other bacterial, fungal or parasitic species. Non-limiting examples of viruses amenable to the present invention include human immunodeficiency viruses (e.g., HIV-1 and HIV-2), influenza viruses (e.g., influenza A, B and C viruses), coronaviruses (e.g., human respiratory coronavirus), hepatitis viruses (e.g., hepatitis viruses A to G), or herpesviruses (e.g., HSV 1-9).

In one embodiment, a nucleic acid array of the present invention includes

a first group of probes, each of which is specific to a different respective Streptococcus pyogenes strain selected from the group consisting of SSI-1, 2F3, Manfredo, MGAS315, MGAS8232 and SF370;

a second group of probes, each of which is specific to a different respective Streptococcus agalactiae strain selected from the group consisting of 2603, A909 and NEM316; and

a third group of probes, each of which is specific to a different respective Staphylococcus epidermidis strain selected from the group consisting of ATCC12228, ATCC14990, O-47, RP62A and SR1.

The nucleic acid array can further include probes that are common to two or more strains of the same species (e.g., Streptococcus pyogenes, Streptococcus agalactiae or Staphylococcus epidermidis). Exemplary polynucleotide probes are depicted in SEQ ID Nos: 18,599 to 605,357. The strain specificity of each of these probes is also provided.

In one example, about 20% to about 40% of perfect match probes on the nucleic acid array can hybridize under stringent or nucleic acid array hybridization conditions to Streptococcus pyogenes transcripts or the complements thereof; about 20% to about 40% of perfect match probes on the nucleic acid array can hybridize under stringent or nucleic acid array hybridization conditions to Streptococcus agalactiae transcripts or the complements thereof; and about 30% to about 50% of perfect match probes on the nucleic acid array can hybridize under stringent or nucleic acid array hybridization conditions to Staphylococcus epidermidis transcripts or the complements thereof.

In another embodiment, a nucleic acid array of the present invention comprises at least 2, 3, 4, 5, 10, 20, 30, 40, 50, 100, 500, 1,000, 2,000, 3,000, 4,000, 5,000, 10,000, 15,000, 18,000 or more polynucleotide probes or probe sets, each of which is capable of hybridizing under stringent or nucleic acid array hybridization conditions to a different respective sequence selected from SEQ ID NOs: 1 to 18,598, or the complement thereof. As used herein, a probe set can hybridize to a sequence if each probe in the probe set can hybridize to the sequence. Each probe set can include any number of probes, such as at least 5, 10, 15, 20, 25 or more.

The present invention contemplates any possible combination of SEQ ID NOs: 1 to 18,598, and any possible combination of probes capable of hybridizing to these sequences or the complements thereof. Many sequences selected from SEQ ID NOs: 1 to 18,598 are intergenic sequences.

In another aspect, the probe arrays of the present invention are protein arrays which comprise:

a first plurality of probes, each of which is specific to a different respective strain of a first species; and

a second plurality of probes, each of which is specific to a different respective strain of a second species.

The protein arrays of the present invention can further include a third plurality of probes, each of which is specific to a different respective strain of a third species. The probes on a protein array of the present invention can be antibodies, antibody mimics, high-affinity binders, or other peptides or protein-binding ligands.

In one embodiment, a protein array of the present invention includes at least 2, 3, 4, 5, 10, 20, 30, 40, 50, 100, 500, 1,000, 2,000, 3,000, 4,000, 5,000, 10,000, 15,000, 18,000 or more probes or probe sets, each of which is capable of binding to a protein encoded by a different respective non-intergenic sequence selected from SEQ ID NOs: 1-18,598, or by a gene that corresponds to that sequence.

The present invention also features methods for developing pharmaceutical compositions for the diagnosis, prophylaxis, or treatment of a non-viral or viral pathogen. The identity of the pathogen can be either known or unknown. In one embodiment, the methods include (1) hybridizing a nucleic acid sample prepared from the pathogen to a nucleic acid array of the present invention; (2) detecting the expression of a virulence or infection-associated gene, or a gene encoding an immunogenic polypeptide; and (3) preparing or selecting a composition capable of eliciting an immunogenic response against the expression product of the gene. In another embodiment, the methods include (1) hybridizing a nucleic acid sample prepared from the pathogen to a nucleic acid array of the present invention; (2) detecting the expression of an antimicrobial resistance gene in the pathogen; and (3) preparing or selecting a treatment which attenuates or eliminates the expression or protein activity of the antimicrobial resistance gene (e.g., by antisense RNA, RNA interference (RNAi) sequences, antibodies, or small molecule inhibitors).

In addition, the present invention features methods for detecting, monitoring, classifying, typing, or quantitating a pathogen of interest in a sample. The methods include the steps of (1) hybridizing nucleic acid molecules prepared from the sample to a nucleic acid array of the present invention, and (2) detecting hybridization signals that are indicative of the presence or absence, gene expression, classification, typing, or quantity of the pathogen in the sample. In one instance, the pathogen being investigated is a β-hemolytic Streptococcus species or a Staphylococcus species.

The present invention further features methods for determining or validating antigen expression of a pathogen of interest. The methods comprise the steps of (1) hybridizing a nucleic acid sample prepared from the pathogen to a nucleic acid array of the present invention; and (2) detecting hybridization signals that are indicative of antigen expression in the pathogen.

Moreover, the present invention features methods for identifying or evaluating agents capable of modulating gene expression in a pathogen of interest. The methods include the steps of (1) contacting an agent with the pathogen; and (2) hybridizing a nucleic acid sample prepared from the pathogen to a nucleic acid array of the present invention, where a change in the hybridization signals after the treatment with the agent, as compared to control hybridization signals, is suggestive of whether the agent can modulate gene expression in the pathogen. In one example, an agent thus identified can inhibit the growth or reduce the virulence of a β-hemolytic Streptococcus species or a Staphylococcus species.

The present invention also features polynucleotide collections comprising at least one polynucleotide capable of hybridizing under stringent or nucleic acid array hybridization conditions to a sequence selected from SEQ ID NOs: 1 to 18,598, or the complement thereof. In addition, the present invention features polypeptide collections comprising at least one polypeptide capable of binding to a protein encoded by a non-intergenic sequence selected from SEQ ID NOs: 1 to 18,598, or by a gene that corresponds to the non-intergenic sequence.

Other features, objects, and advantages of the present invention are apparent in the detailed description that follows. It should be understood, however, that the detailed description, while indicating preferred embodiments of the invention, is given by way of illustration only, not limitation. Various changes and modifications within the scope of the invention will become apparent to those skilled in the art from the detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The drawings are provided for illustration, not limitation.

FIG. 1 shows a hierarchical clustering of 21 Staphylococcus epidermidis strains based on a genotyping study using the nucleic acid array of Example 1.

FIG. 2 illustrates a hierarchical clustering of Group A streptococcus strains based on a genotyping study similar to that in FIG. 1.

FIG. 3 demonstrates a hierarchical clustering of Group B streptococcus strains based on a genotyping study similar to that in FIG. 1.

FIG. 4 shows a hierarchical clustering of strains of Group C or G streptococcus based on a genotyping study similar to that in FIG. 1.

FIG. 5 depicts the distribution of expected present and absent qualifiers for RP62A.

FIG. 6 shows PCR amplification of selected genes.

FIG. 7 indicates the dendrogram and heat map resulting from analysis of the S. epidermidis strains described in Table 2.

FIG. 8 demonstrates the presence or absence of the genes in Table 5 in clinical isolates.

FIG. 9 depicts examples of virulence genes in S. epidermidis.

FIG. 10 is a dendrogram showing DNA similarity between isolates of Group A streptococci (S. pyogenes). In this and subsequent figures, yellow represents a gene present in the strain (positive hybridization signal), blue indicates its absence, and intermediate colors represent intermediate signals indicating a lower than average signal. Each row represents one strain of S. pyogenes; the M type and opacity phenotype (OF⁻ or OF⁺) are given before the strain names. Strains were clustered using normalized signal for all open reading frames on the nucleic acid array of Example 1.

FIG. 11 illustrates classification of S. pyogenes isolates based on the expression of serum opacity factor (SOF). The sof gene is highly variable in sequence and is represented numerous times on the nucleic acid array employed. Some qualifiers represent conserved regions common to more than one gene and some represent unique regions. The existence or nonexistence of the sof gene determines the OF⁺ or OF-phenotype, respectively. Each OF⁺ strain hybridizes to at least one sof qualifier on the array.

FIG. 12 shows the frequency of selected enzyme and exotoxin genes in different S. pyogenes isolates. Each strain is represented by a column and each gene by a row.

FIG. 13 depicts genes whose sequences are conserved among different S. pyogenes isolates. The expression products of these genes are potential vaccine candidates.

### DETAILED DESCRIPTION

The present invention provides probe arrays that allow for concurrent and discriminable detection of multiple strains of different viral or non-viral species. A typical probe array of the present invention includes (1) a first group of probes, each of which is specific to a different respective strain of a first species, and (2) a second group of probes, each of which is specific to a different respective strain of a second species. In many embodiments, a probe array of the present invention further includes at least a third group of probes, each of which is specific to a different respective strain of a third species. A probe array of the present invention can also include probes that are common to two or more different strains of the same species. Examples of non-viral species that are amenable to the present invention include, but are not limited to, bacteria, fungi, parasites, animals, plants, or other prokaryotic or eukaryotic species. Examples of viral species that are amenable to the present invention include, but are not limited to, those selected from the virus families Paramyxoviridae, Adenoviridae, Arenaviridae, Arteriviridae, Bunyaviridae, Caliciviridae, Coronaviridae, Filoviridae, Flaviviridae, Herpesviridae, Orthomyxoviridae, Parvoviridae, Picornaviridae, Poxyiridae, Retroviridae, Reoviridae, Rhabdoviridae, or Togaviridae. In many cases, the non-viral or viral species being investigated are human pathogens.

In one example, a probe array of the present invention comprises at least three different groups of probes. Each probe in the first group is specific to a different corresponding Streptococcus pyogenes strain selected from the group consisting of SSI-1, 2F3, Manfredo, MGAS315, MGAS8232 and SF370; each probe in the second group is specific to a different corresponding Streptococcus agalactiae strain selected from the group consisting of 2603, A909 and NEM316; and each probe in the third group is specific to a different corresponding Staphylococcus epidermidis strain selected from the group consisting of ATCC12228, ATCC14990, O-47, RP62A and SR1.

Different strains of a species typically have different genetic properties. These genetic differences are often manifested in gene expression profiles and therefore become detectable by using the probe arrays of the present invention. The present invention contemplates discriminable detection of different strains that have distinguishable phenotypical characteristics, such as different immunological, morphological, or antibiotic-resistance properties. The present invention also contemplates discriminable detection of strains that have no distinguishable phenotypical properties. As used herein, “strain” includes subspecies.

The following subsections focus on nucleic acid arrays which allow for concurrent and discriminable detection of different strains of Streptococcus pyogenes, Streptococcus agalactiae, and Staphylococcus epidermidis. As appreciated by one of ordinary skill in the art, the same methodology can be readily adapted to making nucleic acid arrays that are suitable for the detection of different strains of other non-viral or viral species. The use of subsections is not meant to limit the invention; each subsection may apply to any aspect of the invention. In this application, the use of “or” means “and/or” unless stated otherwise

A. Identification of Open Reading Frames and Intergenic Sequences

Sequences from different strains of Streptococcus pyogenes, Streptococcus agalactiae, and Staphylococcus epidermidis were collected from publicly available sources (e.g., the microbial genome database at National Center for Biotechnology Information (NCBI), Bethesda, Md. 20894) and from the Pathoseq database (Incyte). These strains included six unique strains of Streptococcus pyogenes (i.e., SSI-1, 2F3, Manfredo, MGAS315, MGAS8232 and SF370), three unique strains of Streptococcus agalactiae (i.e., 2603, A909 and NEM316), and five unique strains of Staphylococcus epidermidis (i.e., ATCC12228, ATCC14990, O-47, RP62A and SR1). Start-site open reading frames (ORFs) were collected as those annotated in public records, or predicted using Glimmer (The Institute for Genomic Research or TIGR), Genemark (the European Bioinformatics Institute), or both. Other custom-designed ORF prediction programs (e.g., a program searching for ATG, GTG or TTG as potential start sites within an open reading frame that encodes a polypeptide having more than 74 amino acids) were also used.

ORFs from each of the two genera (Streptococcus versus Staphylococcus) were separated, and clustered and aligned separately using CAT (Clustering and Alignment Tool) software from DoubleTwist. CAT can cause similar ORFs to cluster together, and then align those similar ORFs to generate one or more sub-clusters. Each sub-cluster of two or more members generates a consensus sequence. The consensus sequences can be generated such that any base ambiguity is identified with the respective IUPAC (International Union of Pure and Applied Chemistry) base representation, which is consistent with the WIPO Standard ST.25 (1998).

The consensus sequences, in addition to all singleton sequences that were either excluded in the initial clustering or sub-clustered into a singleton sub-cluster, were manually curated to verify cluster membership. At this stage, some clusters were joined or separated based on known homologies that were not identified with CAT. In addition, highly repetitive regions in surface proteins were identified and deleted prior to the clustering process.

RNA sequences, such as ribosomal RNAs or tRNAs, were derived from the published RNA sequences associated with Streptococcus pyogenes SSI-1, MGAS315, MGAS8232 and SF370, Streptococcus agalactiae 2603 and NEM316, and Staphylococcus epidermidis ATCC12228, and from additional RNA sequences deposited in Genbank. These sequences were also clustered using the above-described method to generate consensus and singleton sequences.

In addition, intergenic sequences derived from the finished genomes based on the public ORF coordinates and having greater than 50 bases in length were identified and included in the final set of sequences that were used to generate nucleic acid array probes. These finished genomes include Streptococcus pyogenes SSI-1, Manfredo, MGAS315, MGAS8232 and SF370, Streptococcus agalactiae 2603 and NEM316, and Staphylococcus epidermidis ATCC12228 and RP62A. Moreover, a set of sequences from Staphylococcus aureus, mainly representing a collection of genes associated with virulence, were included in the design. The final set of sequences thus produced is collectively referred to as the “parent” sequences and is depicted in SEQ ID NOs: 1 to 18,598.

The first <223> numeric identifier in each sequence listing for SEQ ID NOs: 1 to 18,598 describes the qualifier (e.g., “WAN01UKZO” for SEQ ID NO:1), the type (e.g., “RNA” for SEQ ID NO:1), the source (e.g., “11010011000000” for SEQ ID NO:1), and other relevant information (e.g., “Cluster contains WAN01OVO6:tRNA-Ala:tRNA-Ala:tRNA-Ala:SF370:NC_{—}002737.1” for SEQ ID NO:1) of the corresponding parent sequence. Each category of the above information is separated by semicolons in the <223> numeric identifiers. The qualifier for each parent sequence is generally listed first, and starts with the letters “WAN.” The type of each parent sequence includes RNA, ORF, or intergenic sequence (IG). The source from which each parent sequence was derived is represented by a string of 14 digits (e.g., “11010011000000” for SEQ ID NO:1, “01000000000000” for SEQ ID NO:21, etc.), where each digit signifies a bacterial strain. Specifically, the 1^{st}-14^{th} digits in each string represent Streptococcus pyogenes SF370, Streptococcus pyogenes MGAS315, Streptococcus pyogenes MGAS8232, Streptococcus pyogenes SSI-1, Streptococcus pyogenes Manfredo, Streptococcus pyogenes 2F3, Streptococcus agalactiae 2603, Streptococcus agalactiae NEM316, Streptococcus agalactiae A909, Staphylococcus epidermidis ATCC12228, Staphylococcus epidermidis RP6A, Staphylococcus epidermidis 0-47, Staphylococcus epidermidis ATCC14490, and Staphylococcus epidermidis SR1, respectively. “1” denotes that at least one input sequence for the parent sequence was derived form the corresponding strain, and “0” signifies that no sequence from the corresponding strain contributed to the creation of the parent sequence.

As demonstrated by these 14-digit strings, many parent sequences were derived from two or more strains. Each of these parent sequences had input sequences that are highly conserved among the different strains and therefore can be used for preparing probes that are common to these strains.

As used herein, a polynucleotide probe is “common” to a group of strains if the polynucleotide probe can hybridize under stringent conditions to each and every strain selected from the group. A polynucleotide can hybridize to a strain if the polynucleotide can hybridize to an RNA transcript or genomic sequence of the strain, or the complement thereof. In many embodiments, a probe common to a group of strains can hybridize under stringent conditions to a codon sequence of each strain in the group, or the complement thereof. In many other embodiments, a probe common to a group of strains do not hybridize under stringent conditions to RNA transcripts or genomic sequences of other strains of the same or different species, or the complements thereof.

“Stringent conditions” are at least as stringent as a condition selected from Table 1. In Table 1, hybridization is carried out under the hybridization conditions (Hybridization Temperature and Buffer) for about four hours, followed by two 20-minute washes under the corresponding wash conditions (Wash Temp. and Buffer).

TABLE 1
Stringency Conditions

| Stringency | Polynucleotide | Hybrid | Hybridization | Wash Temp. |
|---|---|---|---|---|
| Condition | Hybrid | Length (bp)¹ | Temperature and Buffer^{H} | and Buffer^{H} |
| A | DNA:DNA | >50 | 65° C.; 1xSSC -or- | 65° C.; 0.3xSSC |
| | | | 42° C.; 1xSSC, 50% formamide | |
| B | DNA:DNA | <50 | T_{B}*; 1xSSC | T_{B}*; 1xSSC |
| C | DNA:RNA | >50 | 67° C.; 1xSSC -or- | 67° C.; 0.3xSSC |
| | | | 45° C.; 1xSSC, 50% formamide | |
| D | DNA:RNA | <50 | T_{D}*; 1xSSC | T_{D}*; 1xSSC |
| E | RNA:RNA | >50 | 70° C.; 1xSSC -or- | 70° C.; 0.3xSSC |
| | | | 50° C.; 1xSSC, 50% formamide | |
| F | RNA:RNA | <50 | T_{F}*; 1xSSC | T_{f}*; 1xSSC |
| G | DNA:DNA | >50 | 65° C.; 4xSSC -or- | 65° C.; 1xSSC |
| | | | 42° C.; 4xSSC, 50% formamide | |
| H | DNA:DNA | <50 | T_{H}*; 4xSSC | T_{H}*; 4xSSC |
| I | DNA:RNA | >50 | 67° C.; 4xSSC -or- | 67° C.; 1xSSC |
| | | | 45° C.; 4xSSC, 50% formamide | |
| J | DNA:RNA | <50 | T_{J}*; 4xSSC | T_{J}*; 4xSSC |
| K | RNA:RNA | >50 | 70° C.; 4xSSC -or- | 67° C.; 1xSSC |
| | | | 50° C.; 4xSSC, 50% formamide | |
| L | RNA:RNA | <50 | T_{L}*; 2xSSC | T_{L}*; 2xSSC |
| ¹The hybrid length is that anticipated for the hybridized region(s) of the hybridizing polynucleotides. When hybridizing a polynucleotide to a target polynucleotide of unknown sequence, the hybrid length is assumed to be that of the hybridizing polynucleotide. When polynucleotides of known sequence are hybridized, the hybrid length can be determined by aligning the sequences of the polynucleotides and identifying the region or regions of optimal sequence complementarity. | | | | |
| ^{H}SSPE (1xSSPE is 0.15M NaCl, 10 mM NaH₂PO₄, and 1.25 mM EDTA, pH 7.4) can be substituted for SSC (1xSSC is 0.15M NaCl and 15 mM sodium citrate) in the hybridization and wash buffers. | | | | |
| T_{B}* − T_{R}*: The hybridization temperature for hybrids anticipated to be less than 50 base pairs in length should be 5-10° C. less than the melting temperature (Tₘ) of the hybrid, where Tₘ is determined according to the following equations. For hybrids less than 18 base pairs in length, Tₘ(° C.) = 2(# of A + T bases) + 4(# of G + C bases). For hybrids between 18 | | | | |
| # and 49 base pairs in length, Tₘ(° C.) = 81.5 + 16.6(log₁₀Na⁺) + 0.41(% G + C) − (600/N), where N is the number of bases in the hybrid, and Na⁺ is the molar concentration of sodium ions in the hybridization buffer (Na⁺ for 1xSSC = 0.165M). | | | | |

The 14-digit strings in the first <223> numeric identifiers of SEQ ID NOs: 1 to 18,598 also illustrate that some parent sequences were derived from only one bacterial strain. Many of these parent sequences are singleton sequences which are unique to only one of the Streptococcus pyogenes, Streptococcus agalactiae or Staphylococcus epidermidis strains that are being investigated. Many of these sequences can be used to prepare probes that are specific to the corresponding strains from which the sequences were derived. Some singleton sequences, however, are present in more than one genomes, but were not identified as ORFs and, therefore, were not in the input sequence set.

As used herein, a polynucleotide probe is “specific” to a strain selected from a group of strains if the polynucleotide probe can hybridize under stringent conditions to an RNA transcript or genomic sequence of the strain, or the complement thereof, but not to RNA transcripts or genomic sequences of other strains in the group, or the complements thereof. In many embodiments, a probe specific to a strain can hybridize under stringent conditions to a codon sequence of the strain, or the complement thereof.

As appreciated by one of ordinary skill in the art, ORFs and other expressible or intergenic sequences can be similarly extracted from other strains of Streptococcus pyogenes, Streptococcus agalactiae or Staphylococcus epidermidis, or from strains of other Staphylococcus or Streptococcus species. Examples of other Staphylococcus or Streptococcus species include, but are not limited to, Staphylococcus aureus, Staphylococcus saprophyticus, Staphylococcus haemolyticus, Staphylococcus hominis, Group C streptococci (beta hemolytic, occasionally alpha or gamma, e.g., Streptococcus anginosus or Streptococcus equismilis), Group D streptococci (alpha or gamma hemolytic, occasionally beta, e.g., Streptococcus bovis), Group E streptococci, Group F streptococci (beta hemolytic, e.g., Streptococcus anginosus), Group G streptococci (beta hemolytic, e.g., Streptococcus anginosus), Groups H and K through V streptococci, Viridans streptococci (e.g., Streptococcus mutans or Streptococcus sangui), Streptococcus faecalis and Streptococcus pneumoniae.

Other non-viral or viral species can also be used to extract consensus or singleton sequences. Probes common to two or more strains of these non-viral or viral species, or probes specific to a particular strain, can be derived from the consensus or singleton sequences, respectively. Non-viral species amenable to the present invention include, but are not limited to, bacterial species selected from Actinobacillus (e.g., Actinobacillus lignieresi, Actinobacillus pleuropneumoniae), Actinomyces (e.g., Actinomyces bovis, Actinomyces israelii or Actinomyces naeslundii), Aerobacter (e.g., Aerobacter aerogenes), Alloiococcus (e.g., Alloiococcus otitidis) Anaplasma (e.g., Anaplasma marginale), Bacillus (e.g., Bacillus anthracis or Bacillus cereus), Bordetella (e.g., Bordetella pertussis or Bordetella parapertussis), Borrelia (e.g., Borrelia anserina, Borrelia recurrentis or Borrelia burgdorferi), Brucella (e.g., Brucella canis or Brucella melintensis), Campylobacter (e.g., Campylobacter jejuni), Chlamydia (e.g., Chlamydia psittaci, Chlamydia pneumoniae, Chiamydia trachomatis), Clostridium (e.g., Clostridium botulinum, Clostridium chauvoei, Clostridium difficile, Clostridium hemolyticium, Clostridium novyi, Clostridium perfringens, Clostridium septicum or Clostridium tetani), Corynebacterium (e.g., Corynebacterium equi, Corynebacterium diphtheriae, Corynebacterium pyogenes or Corynebacterium renale), Coxiella (e.g., Coxiella burneti), Cowdria (e.g., Cowdria ruminantium), Dermatophilus (e.g., Dermatophilus congolensis), Erysipelothrix (e.g., Erysipelothrix insidiosa or Erysipelothrix rhusopathiae), Escherichia (e.g., Escherichia coli), Francisella (e.g., Francisella tularenssis), Fusiformis (e.g., Fusiformis necrophorus), Haemobartonella (e.g., Haemobartonella canis), Haemophilus (e.g., Haemophilus influenza, both typable and nontypable, or Haemophilus parainfluenzae), Helicobacter (e.g., Helicobacter pylori) Klebsiella (e.g., Klebsiella pneumoniae), Legionella (e.g., Legionella pneumophila), Leptospira (e.g., Leptospira interrogans), Listeria (e.g., Listeria monocytogenes), Moraxella (e.g., Moraxella bovis or Moraxella catarrhalis), Mycobacterium (e.g., Mycobacterium bovis, Mycobacterium leprae or Mycobacterium tuberculosis), Mycoplasma (e.g., Mycoplasma hyopneumoniae, Mycoplasma gallisepticum or Mycoplasma pneumoniae), Nanophyetus (e.g., Nanophyetus salmincola), Neisseria (e.g., Neisseria gonorrhoeae or Neisseria meninigitidis), Nocardia (e.g., Nocardia asteroides), Pasteurella (e.g., Pasteurella anatipestifer, Pasteurella haemolytica or Pasteurella multocida), Proteus (e.g., Proteus vulgaris or Proteus mirabilis) Pseudomonas (e.g., Pseudomonas aeruginosa), Rickettsia (e.g., Rickettsia mooseria, Rickettsia prowazekii, Rickettsia rickettsii or Rickettsia tsutsugamushi), Salmonella (e.g., Salmonella typhi or Salmonella typhimurium), Shigella (e.g., Shigella dysenteriae or Shigella boydii), Treponema (e.g., Treponema pallidum), Vibrio (e.g., Vibrio cholerae), or Yersinia (e.g., Yersinia enterocolitica or Yersinia pestis); protozoan species selected from Eimeria, Anaplasma, Giardia, Babesia, Trichomonas, Entamoeba, Balantidium, Plasmodium, Leishmania, Toxoplasma, Trypanosoma, Entamoeba, Trichomonas, Toxoplasmosa, or Pneumocystis; fungal species selected from Blastomyces, Microsporum, Aspergillis, Candida, Coccidiodes, Cryptococcus, Histoplasma or Trichophyton; and parasites such as trypanosomes, tapeworms, roundworms, and helminthes. Non-limiting examples of viral species that are amenable to the present invention include Paramyxoviridae (e.g., pneumovirus, morbillivirus, metapneumovirus, respirovirus or rubulavirus), Adenoviridae (e.g., adenovirus), Arenaviridae (e.g., arenavirus such as lymphocytic choriomeningitis virus), Arteriviridae (e.g., porcine respiratory and reproductive syndrome virus or equine arteritis virus), Bunyaviridae (e.g., phlebovirus or hantavirus), Caliciviridae (e.g., Norwalk virus), Coronaviridae (e.g., coronavirus or torovirus), Filoviridae (e.g., Ebola-like viruses), Flaviviridae (e.g., hepacivirus or flavivirus), Herpesviridae (e.g., simplexvirus, varicellovirus, cytomegalovirus, roseolovirus, or lymphocryptovirus), Orthomyxoviridae (e.g., influenza A virus, influenza B virus, influenza C virus, or thogotovirus), Parvoviridae (e.g., parvovirus), Picornaviridae (e.g., enterovirus or hepatovirus), Poxyiridae (e.g., orthopoxvirus, avipoxvirus, or leporipoxvirus), Retroviridae (e.g., lentivirus or spumavirus), Reoviridae (e.g., rotavirus), Rhabdoviridae (e.g., lyssavirus, novirhabdovirus, or vesiculovirus), and Togaviridae (e.g., alphavirus or rubivirus). Sequences from other infectious or pathogenic microbes can also be collected to make the probe arrays of the present invention.

B. Preparation of Polynucleotide Probes for Detecting Streptococcus pyogenes, Streptococcus agalactiae or Staphylococcus epidermidis Strains

The parent sequences depicted in SEQ ID NOs: 1-18,598 can be used to prepare polynucleotide probes. The probes for each parent sequence can hybridize under stringent or nucleic acid array hybridization conditions to that parent sequence, or the complement thereof. In many embodiments, the probes for each parent sequence are incapable of hybridizing under stringent or nucleic acid array hybridization conditions to other parent sequences, or the complements thereof. In one example, the probes for each parent sequence comprise or consist of an unambiguous sequence fragment of the parent sequence, or the complement thereof.

As used herein, “nucleic acid array hybridization conditions” refer to the temperature and ionic conditions that are normally used in nucleic acid array hybridization. In many examples, these conditions include 16-hour hybridization at 45° C., followed by at least three 10-minute washes at room temperature. The hybridization buffer comprises 100 mM MES, 1 M [Na⁺], 20 mM EDTA, and 0.01% Tween 20. The pH of the hybridization buffer can range between 6.5 and 6.7. The wash buffer is 6×SSPET. 6×SSPET contains 0.9 M NaCl, 60 mM NaH₂PO₄, 6 mM EDTA, and 0.005% Triton X-100. Under more stringent nucleic acid array hybridization conditions, the wash buffer can contain 100 mM MES, 0.1 M [Na⁺], and 0.01% Tween 20. See also GENECHIP® EXPRESSION ANALYSIS TECHNICAL MANUAL (701021 rev. 3, Affymetrix, Inc. 2002), which is incorporated herein by reference in its entirety.

The nucleic acid probes of the present invention can be DNA, RNA, or PNA (“Peptide Nucleic Acid”). Other modified forms of DNA, RNA, or PNA can also be used. The nucleotide units in each probe can be either naturally occurring residues (such as deoxyadenylate, deoxycytidylate, deoxyguanylate, deoxythymidylate, adenylate, cytidylate, guanylate, and uridylate), or synthetically produced analogs that are capable of forming desired base-pair relationships. Examples of these analogs include, but are not limited to, aza and deaza pyrimidine analogs, aza and deaza purine analogs, and other heterocyclic base analogs, wherein one or more of the carbon and nitrogen atoms of the purine and pyrimidine rings are substituted by heteroatoms, such as oxygen, sulfur, selenium, and phosphorus. Similarly, the polynucleotide backbones of the probes of the present invention can be either naturally occurring (such as through 5′ to 3′ linkage), or modified. For instance, the nucleotide units can be connected via non-typical linkage, such as 5′ to 2′ linkage, so long as the linkage does not interfere with hybridization. For another instance, peptide nucleic acids, in which the constitute bases are joined by peptide bonds rather than phosphodiester linkages, can be used.

In one embodiment, the nucleic acid probes of the present invention have relatively high sequence complexity. In many examples, the probes do not contain long stretches of the same nucleotide. In addition, the probes may be designed such that they do not have a high proportion of G or C residues at the 3′ ends. In another embodiment, the probes do not have a 3′ terminal T residue. Depending on the type of assay or detection to be performed, sequences that are predicted to form hairpins or interstrand structures, such as “primer dimers,” can be either included in or excluded from the probe sequences. In many embodiments, each probe employed in the present invention does not contain any ambiguous base.

Any part of a parent sequence can be used to prepare probes. Multiple probes, such as 5, 10, 15, 20, 25, 30, or more, can be prepared for each parent sequence. These multiple probes may or may not overlap each other. Overlap among different probes may be desirable in some assays.

In many embodiments, the probes for a parent sequence have low sequence identities with other parent sequences, or the complements thereof. For instance, each probe for a parent sequence can have no more than 70%, 60%, 50% or less sequence identity with other parent sequences, or the complements thereof. This reduces the risk of undesired cross-hybridization. Sequence identity can be determined using methods known in the art. These methods include, but are not limited to, BLASTN, FASTA, and FASTDB. The Genetics Computer Group (GCG) program can also be used, which is a suite of programs including BLASTN and FASTA.

The suitability of the probes for hybridization can be evaluated using various computer programs. Suitable programs for this purpose include, but are not limited to, LaserGene (DNAStar), Oligo (National Biosciences, Inc.), MacVector (Kodak/IBI), and the standard programs provided by the GCG.

Any method or software program known in the art may be used to prepare probes for the parent sequences of the present invention. In one embodiment, polynucleotide probes are generated by using Array Designer, a software package provided by TeleChem International, Inc (Sunnyvale, Calif. 94089). Examples of the polynucleotide probes thus generated are depicted in SEQ ID NOs: 18,599 to 605,357. The <223> numeric identifier for each of these probes (as well as SEQ ID NOs: 605,358 to 1,276,209, see infra) provides the SEQ ID number and the qualifier of the corresponding parent sequence, as well as the start and stop positions of the probe in the corresponding parent sequence and the specificity of the probe to different Streptococcus pyogenes, Streptococcus agalactiae and Staphylococcus epidermidis strains. Each category of these information is separated by semicolons in the <223> numeric identifiers. For instance, for probe SEQ ID NO: 18,599, the corresponding parent sequence to which the probe or the complement thereof can hybridize has “SEQ ID NO:4” and qualifier “WAN01UL4E.” The probe starts at residue 11 (“Start 11”) and ends at residue 35 (‘Stop 35”) in the parent sequence.

The specificity of each probe in SEQ ID NOs: 18,599 to 605,357 (as well as SEQ ID NOs: 605,358 to 1,276,209, see infra) is represented by a 15-digit string (e.g., “111111111100000” for SEQ ID NO: 18,599). Each digit in the string denotes whether the probe has a hit against the genome of the corresponding strain (both the forward strand and the reverse complement). The 1^{st}-15^{th} digits in each string represent Streptococcus pyogenes SF370, Streptococcus pyogenes MGAS315, Streptococcus pyogenes MGAS8232, Streptococcus pyogenes SSI-1, Streptococcus pyogenes Manfredo, Streptococcus pyogenes 2F3, Streptococcus pyogenes M6, Streptococcus agalactiae 2603, Streptococcus agalactiae NEM316, Streptococcus agalactiae A909, Staphylococcus epidermidis ATCC12228, Staphylococcus epidermidis RP62A, Staphylococcus epidermidis 0-47, Staphylococcus epidermidis ATCC14990, and Staphylococcus epidermidis SR1, respectively. “1” on each digit signifies that the probe was found at least once in the corresponding genome being searched, and “0” indicates that no hit was produced when the probe sequence was searched against the corresponding genome. Incomplete genomes were used for Streptococcus pyogenes 2F3, Streptococcus agalactiae A909, Staphylococcus epidermidis ATCC14990 and Staphylococcus epidermidis SR1 in determining each probe's specificity with respect to these strains.

Many probes in SEQ ID NOs: 18,599 to 1,276,209 are shared by two or more strains. These probes can be used as common probes for the detection of each of these shared strains. Many other probes in SEQ ID NOs: 18,599 to 1,276,209 are unique to only one strain and, therefore, can be used to specifically detect that strain.

In many embodiments, perfect mismatch probes are prepared for each probe depicted in SEQ ID NOs: 18,599 to 605,357. A perfect mismatch probe has the same sequence as the corresponding perfect match probe except for a homomeric substitution (i.e., A to T, T to A, G to C, or C to G) at or near the center of the perfect mismatch probe. For instance, if the perfect match probe has 2n nucleotide residues, the homomeric substitution in the corresponding perfect mismatch probe is either at the n or n+1 position, but not at both positions. If the perfect match probe has 2n+1 nucleotide residues, the homomeric substitution in the corresponding perfect mismatch probe is at the n+1 position.

The polynucleotide probes of the present invention can be synthesized using a variety of methods. Examples of these methods include, but are not limited to, automated or high throughput DNA synthesizers, such as those provided by Millipore, GeneMachines, or BioAutomation. In many embodiments, the synthesized probes are substantially free of impurities. In many other embodiments, the probes are substantially free of other contaminants that may hinder the desired functions of the probes. The probes can be purified or concentrated using numerous methods, such as reverse phase chromatography, ethanol precipitation, gel filtration, electrophoresis, or a combination thereof.

The parent sequences or the polynucleotide probes of the present invention can be used to detect, identify, distinguish, classify, type, validate antigen expression, or quantitate different strains of streptococci (such as Streptococcus pyogenes, Streptococcus agalactiae or other β-hemolytic streptococci) or Staphylococcus spp. (such as Staphylococcus epidermidis or Staphylococcus aureus) in a sample of interest. Methods suitable for this purpose include, but are not limited to, nucleic acid arrays (including bead arrays), Southern Blot, Northern Blot, PCR, and RT-PCR. A sample of interest can be, without limitation, a food sample, an environmental sample, a pharmaceutical sample, a bacterial culture, a clinical sample, a chemical sample, or a biological sample. Non-limiting examples of suitable biological samples include body fluid samples, including blood or its components (e.g., plasma or serum), menses, mucous, sweat, tears, urine, feces, saliva, sputum, semen, uro-genital secretions, gastric washes, pericardial or peritoneal fluids or washes, a throat swab, pleural washes, ear wax, hair, skin cells, nails, mucous membranes, amniotic fluid, vaginal secretions or other secretions from the body, spinal fluid, human breath, gas samples containing body odors, flatulence or other gases, any biological tissue or matter, or an extractive or suspension of any of these.

As appreciated by those skilled in the art, parent sequences can be similarly isolated from the genomic sequences of other non-viral or viral strains or species. These parent sequences include ORFs, intergenic sequences, or other transcribable or non-transcribable elements. Polynucleotide probes for these parent sequences can be similarly prepared using the above-described methods.

C. Nucleic Acid Arrays

The polynucleotide probes of the present invention can be used to make nucleic acid arrays which allow for concurrent and discriminable detection of multiple strains of different species. In many embodiments, the nucleic acid arrays of the present invention include at least one substrate support which has a plurality of discrete regions. The location of each discrete region is either known or determinable. These discrete regions can be organized in various forms or patterns. For instance, the discrete regions can be arranged as an array of regularly spaced areas on a surface of the substrate. Other regular or irregular patterns, such as linear, concentric or spiral patterns, can also be used.

Polynucleotide probes can be stably attached to respective discrete regions through covalent or non-covalent interactions. As used herein, a polynucleotide probe is “stably” attached to a discrete region if the polynucleotide probe retains its position relative to the discrete region during nucleic acid array hybridization.

A variety of methods can be used to attach polynucleotide probes to a nucleic acid array of the present invention. In one embodiment, polynucleotide probes are covalently attached to a substrate support by first depositing the polynucleotide probes to respective discrete regions on a surface of the substrate support and then exposing the surface to a solution of a cross-linking agent, such as glutaraldehyde, borohydride, or other bifunctional agents. In another embodiment, polynucleotide probes are covalently bound to a substrate via an alkylamino-linker group or by coating a substrate (e.g., a glass slide) with polyethylenimine followed by activation with cyanuric chloride for coupling the polynucleotides. In yet another embodiment, polynucleotide probes are covalently attached to a nucleic acid array through polymer linkers. The polymer linkers may improve the accessibility of the probes to their purported targets. In many cases, the polymer linkers do not significantly interfere with the interactions between the probes and their purported targets.

Polynucleotide probes can also be stably attached to a nucleic acid array through non-covalent interactions. In one embodiment, polynucleotide probes are attached to a substrate support through electrostatic interactions between positively charged surface groups and the negatively charged probes. In another embodiment, a substrate employed in the present invention is a glass slide having a coating of a polycationic polymer on its surface, such as a cationic polypeptide. The polynucleotide probes are bound to these polycationic polymers. In yet another embodiment, the methods described in U.S. Pat. No. 6,440,723, which is incorporated herein by reference, are used to stably attach polynucleotide probes to a nucleic acid array of the present invention.

Numerous materials can be used to make the substrate supports. Suitable materials include, but are not limited to, glass, silica, ceramics, nylon, quartz wafers, gels, metals, and paper. A substrate support can be flexible or rigid. In one embodiment, a substrate support is in the form of a tape that is wound up on a reel or cassette. A nucleic acid array can include two or more substrate supports. In many embodiments, the substrate supports are non-reactive with reagents that are used in nucleic acid array hybridization.

The surface(s) of a substrate support can be smooth and substantially planar. The surface(s) of a substrate support can also have a variety of configurations, such as raised or depressed regions, trenches, v-grooves, mesa structures, or other regular or irregular configurations. The surface(s) of the substrate can be coated with one or more modification layers. Suitable modification layers include inorganic or organic layers, such as metals, metal oxides, polymers, or small organic molecules. In one embodiment, the surface(s) of the substrate is chemically treated to include groups such as hydroxyl, carboxyl, amine, aldehyde, or sulfhydryl groups.

The discrete regions on a nucleic acid array of the present invention can be of any size, shape and density. For instance, they can be squares, ellipsoids, rectangles, triangles, circles, or other regular or irregular geometric shapes, or a portion or combination thereof. In one embodiment, each discrete region has a surface area of less than 10⁻¹ cm², such as less than 10^{-2, 10}^{-3, 10}⁻⁴, 10⁻⁵, 10⁻⁶, or 10⁻⁷ cm². In another embodiment, the spacing between each discrete region and its closest neighbor, measured from center-to-center, is in the range of from about 10 to about 400 μm. The density of the discrete regions can range, for example, from 50 to 50,000 regions/cm².

A variety of methods can be used to make the nucleic acid arrays of the present invention. For instance, the probes can be synthesized in a step-by-step manner on a substrate, or can be attached to a substrate in pre-synthesized forms. Algorithms for reducing the number of synthesis cycles can be used. In one embodiment, a nucleic acid array of the present invention is synthesized in a combinational fashion by delivering monomers to the discrete regions through mechanically constrained flowpaths. In another embodiment, a nucleic acid array of the present invention is synthesized by spotting monomer reagents onto a substrate support using an ink jet printer (such as the DeskWriter C manufactured by Hewlett-Packard). In yet another embodiment, polynucleotide probes are immobilized on a nucleic acid array by using photolithography techniques.

Bead arrays and other types of biochips are also contemplated by the present invention. A bead array comprises a plurality of beads, with each bead stably associated with one or more polynucleotide probes of the present invention.

In one embodiment, a nucleic acid array of the present invention includes at least three different groups of probes: each probe in the first group is specific to a different respective Streptococcus pyogenes strain selected from SSI-1, 2F3, Manfredo, MGAS315, MGAS8232 and SF370; each probe in the second group is specific to a different respective Streptococcus agalactiae strain selected from the group consisting of 2603, A909 and NEM316; and each probe in the third group is specific to a different respective Staphylococcus epidermidis strain selected from the group consisting of ATCC12228, ATCC14990, O-47, RP62A and SR1. Exemplary probes suitable for this nucleic acid array can be selected from SEQ ID NOs: 18,599 to 605,357.

In another embodiment, a nucleic acid array of the present invention further includes at least 1, 2, 3, 4, 5, 6, 7, 8, 9, 10 or more polynucleotide probes or probe sets, each of which is common to two or more strains of a non-viral or viral species. For instance, the nucleic acid array can include at least 3 polynucleotide probes: the first probe is common to two or more Streptococcus pyogenes strains selected from SSI-1, 2F3, Manfredo, MGAS315, MGAS8232 and SF370; the second probe is common to two or more Streptococcus agalactiae strains selected from the group consisting of 2603, A909 and NEM316; and the third probe is common to two or more Staphylococcus epidermidis strains selected from the group consisting of ATCC12228, ATCC14990, O-47, RP62A and SR1. Probes suitable for this purpose can also be selected from SEQ ID NOs: 18,599 to 605,357.

In still another embodiment, a nucleic acid array of the present invention includes at least 2, 3, 4, 5, 10, 20, 50, 100, 200 or more different probes or probe sets, each of which is specific to the same strain. These probes or probe sets can be positioned in the same or different discrete regions on the nucleic acid array. As used herein, two polynucleotides are “different” if they have different nucleic acid sequences.

In yet another embodiment, a nucleic acid array of the present invention includes at least 1, 2, 5, 10, 20, 30, 40, 50, 100, 200, 500, 1,000, 2,000, 3,000, 4,000, 5,000, 10,000, 15,000, 18,000 or more different probes or probe sets, each of which can hybridize under stringent or nucleic acid array hybridization conditions to a different respective sequence selected from SEQ ID NOs: 1 to 18,598, or the complement thereof.

In one example, the nucleic acid array includes at least two groups of probes. Each group of probes can hybridize under stringent or nucleic acid array hybridization conditions to a different group of sequences selected from the following groups:

Group 1: SEQ ID NOs: 1-5,840 (derived from Streptococcus pyogenes) or the complements thereof;

Group 2: SEQ ID NOs: 5,841-10,822 (derived from Streptococcus agalactiae) or the complements thereof;

Group 3: SEQ ID NOs: 10,823-18,217 (derived from Staphylococcus epidermidis) or the complements thereof; and

Group 4: SEQ ID NOs: 18,218-18,598 (derived from Staphylococcus aureus) or the complements thereof.

In another example, the nucleic acid array includes at least three groups of probes, where the first group of probes can hybridize under stringent or nucleic acid array hybridization conditions to sequences selected from Group 1; the second group of probes can hybridize under stringent or nucleic acid array hybridization conditions to sequences selected from Group 2; and the third group of probes can hybridize under stringent or nucleic acid array hybridization conditions to sequences selected from Group 3. The nucleic acid array may further include a fourth group of probes capable of hybridizing under stringent or nucleic acid array hybridization conditions to sequences selected from Group 4. Each group of probes can include at least 1, 2, 3, 4, 5, 10, 50, 100, 500, 1,000 or more polynucleotide probes, each of which can hybridize to a different respective sequence selected from SEQ ID NOs: 1 to 18,598, or the complement thereof. Non-limiting examples of probes suitable for this purpose can be selected from SEQ ID NOs: 18,599 to 605,357.

In yet another embodiment, a nucleic acid array of the present invention includes each and every probe selected from SEQ ID NOs: 18,599 to 605,357.

The length of each probe employed in the present invention can be selected to achieve the desired hybridization effect. For instance, a probe can include or consist of about 15, 20, 25, 30, 35, 40, 45, 50, 60, 70, 80, 90, 100, 200, 300, 400 or more consecutive nucleotides.

Multiple probes for the same gene can be included in a nucleic acid array of the present invention. For instance, at least 2, 5, 10, 15, 20, 25, 30 or more different probes can be used to detect the same gene. Each of these different probes can be attached to a different respective region on the nucleic acid array. Alternatively, two or more different probes can be attached to the same discrete region. The concentration of one probe with respect to the other probe or probes in the same discrete region may vary according to the objectives and requirements of the particular experiment. In one embodiment, different probes in the same region are present in approximately equimolar ratio.

Probes for different genes are typically attached to different respective regions on a nucleic acid array. In certain applications, probes for different genes are attached to the same discrete region.

In one embodiment, a nucleic acid array of the present invention includes probes for virulence or antimicrobial resistance genes. The virulence or resistance genes may be unique for a particular bacterial strain, or shared by several bacterial strains. Examples of virulence genes include, but are not limited to, various toxin and pathogenicity factor genes, such as those encoding immunoglobulin-binding proteins, serum opacity factor, M protein, C5a peptidase, Fc-binding proteins, collagenase, hyaluronate lyase, streptococcal pyrogenic exotoxins, mitogenic factor, alpha C protein, fibrinogen binding protein, fibronectin binding protein, coagulase, enterotoxins, exotoxins, leukocidins, or V8 protease. Examples of antimicrobial resistance genes include, but are not limited to, penicillin-resistance genes, tetracycline-resistance genes, streptomycin-resistance genes, methicillin-resistance genes, and glycopeptide drug-resistance genes.

In one example, a nucleic acid array of the present invention includes polynucleotide probes capable of hybridizing to one or more qualifiers selected from Tables 5, 6, or 7. For instance, the nucleic acid array can include 1, 2, 3, 4, 5, 6, 7, 8, 10, or more polynucleotide probes, each of which can hybridize to a different qualifier selected from Tables 5, 6, or 7. These qualifiers can be selected from the same table or different tables. The present invention contemplates any combination of the qualifiers selected from Tables 5, 6, or 7. As used herein, a probe is capable of hybridizing to a qualifier if the probe can hybridize under stringent or nucleic acid array hybridization conditions to the parent sequence of the qualifier, or the complement of that parent sequence. Exemplary probes suitable for this purpose are described in SEQ ID NOs: 18,599 to 605,357.

The present invention also features nucleic acid arrays which comprise polynucleotide probes capable of hybridizing to one or more genes selected from Tables 5, 6, or 7. The present invention contemplates any combination of the genes selected from Tables 5, 6, or 7. A probe is capable of hybridizing to a gene if the probe can hybridize under stringent or nucleic acid array hybridization conditions to the DNA, or the complement thereof, of the gene. In many cases, the probe is also capable of hybridizing under stringent or nucleic acid array hybridization conditions to the RNA transcript, or the complement thereof, of the gene.

In another embodiment, a nucleic acid array of the present invention comprises probes for infection-related genes or qualifiers. These genes or qualifiers are expressed, or exist, in the majority of infectious strains, but have less frequency in non-infectious strains (e.g., no more than 50%, 25%, 10%, 5%, or 1% of non-infectious strains express these genes). Non-limiting examples of these genes or qualifiers are depicted in Table 8. The present invention contemplates any combination of the genes (or qualifiers) selected from Table 8. For instance, a nucleic acid array can comprise at least 1, 2, 3, 4, 5, 6, 7, 8, 9, 10, or more polynucleotide probes, each of which is capable of hybridizing to a different qualifier selected from Table 8. In one example, the nucleic acid array comprises polynucleotide probes for all of the qualifiers (or genes) depicted in Table 8.

The nucleic acid arrays of the present invention can also include control probes which can hybridize under stringent or nucleic acid array hybridization conditions to respective control sequences, or the complements thereof. Exemplary control sequences are depicted in SEQ ID NOs: 82,738-82,806 of U.S. patent application Ser. No. 10/859,198 entitled “Nucleic Acid Arrays for Detecting Multiple Strains of A Non-Viral Species” and filed Jun. 3, 2004 (by William M. Mounts et al.), and exemplary probes for these control sequences are described in SEQ ID NOs: 280,086-282,011 of the same application. All of these control sequences and probes are incorporated herein by reference.

The nucleic acid arrays of the present invention can further include mismatch probes as controls. In many instances, the mismatch residue in each mismatch probe is located near the center of the probe such that the mismatch is more likely to destabilize the duplex with the target sequence under the hybridization conditions. In one embodiment, each mismatch probe on a nucleic acid array of the present invention is a perfect mismatch probe, and is stably attached to a discrete region different from that of the corresponding perfect match probe.

D. Applications

The nucleic acid arrays of the present invention can be used to monitor, type, or classify different clinically important strains, allowing epidemic strains to be promptly identified during outbreaks. The nucleic acid arrays of the present invention also allow different strains to be typed according to their responses to specific genes, therefore replacing immunological methods (e.g., M protein of Streptococcus pyogenes). Furthermore, the nucleic acid arrays of the present invention can be used to identify specific virulence markers on a particular strain or species. The presence of specific virulence markers is frequently associated with particular forms of invasive disease.

The genetic variability or genotype of a pathogen is often of relevance in the development of suitable immunization or treatment strategies. For instance, the presence of β-lactamase gene in a bacterium is often indicative of bacterial resistance to β-lactam antibiotics. As a result, a β-lactamase inhibitor can be employed in combination with antibiotics to treat infections caused by the bacterium (e.g., ZOSYN® of Wyeth, which includes piperacillin (a semisynthetic penicillin) and tazobactam (a β-lactamase inhibitor)). For another instance, the identification of expression of a gene that encodes an immunogenic surface protein often facilitates the design or selection of antigens for inclusion in an efficacious immunogenic composition against the corresponding pathogen.

The nucleic acid arrays of the present invention allow the genotyping of different pathogens in one single experimental setup. The nucleic acid arrays of the present invention also allow the analysis of a particular sample or isolate for the presence of specific virulence, antimicrobial resistance or infection-associated genes, or genes encoding specific immunogenic surface proteins, thereby facilitating rapid selection of efficacious immunogenic compositions or treatments during outbreaks. Methods suitable for this purpose typically comprise:

preparing a nucleic acid sample from a sample of interest;

hybridizing the nucleic acid sample to a nucleic acid array of the present invention; and

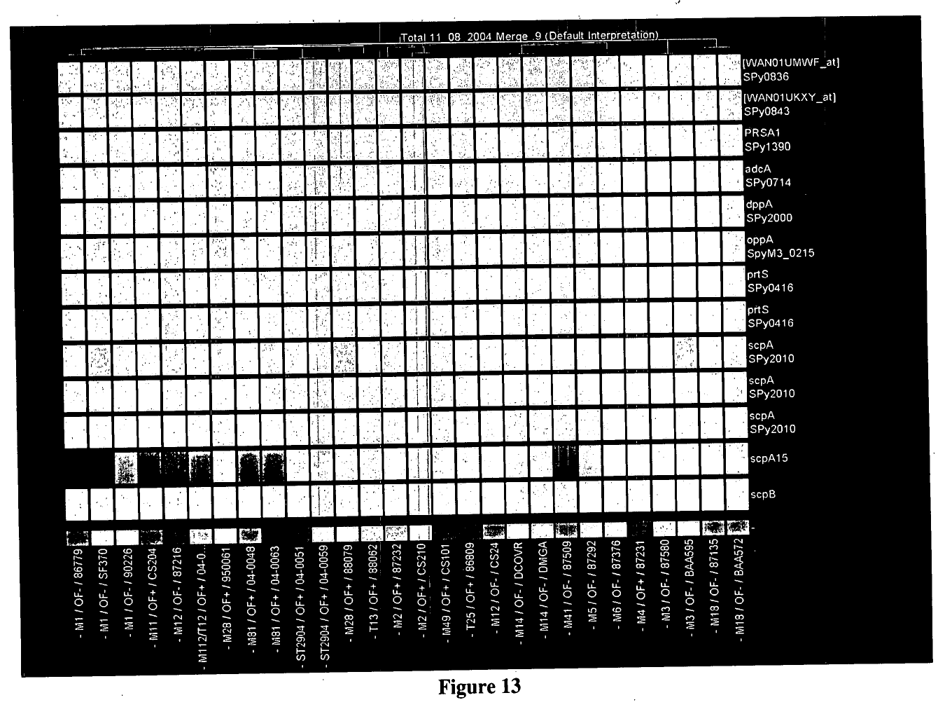

detecting hybridization signals on the nucleic acid array to determine the existence or nonexistence (or expression or non-expression) of a gene of interest. In many embodiments, the sample of interest is a biological or environmental sample, and the nucleic acid sample prepared therefrom is a DNA or RNA sample. The gene being investigated can be a virulence gene, an antimicrobial resistance gene, an infection-associated gene, or a gene encoding a conserved surface antigen. Non-limited examples of nucleic acid arrays suitable for this purpose include the above-described arrays which comprise probes for the genes or qualifiers selected from Tables 5-8. The nucleic acid arrays can also include probes for one or more genes or qualifiers selected from FIG. 13, e.g., WAN01UMWF_at (SPy0836), WAN01UKXY_at (SPy0843), WAN01UNE5_at (PRSA1), WAN01UK2H_at (adcA), WAN01UKQ6_at (dppA), WAN01UMZE_at (oppA), WAN01UJHC-seg1_at (prtS), WAN01UJHC-seg2_at (prtS), WAN01UMYS_at (scpA), WAN1UMYU_at (scpA), WAN01UMYR_at (scpA), WAN01UMCN_at (scpA15), or WAN01UMSZ_at (scpB). The present invention contemplates any combination of the genes or qualifiers selected from FIG. 13.

In addition, due to gene conservation, a nucleic acid array of the present invention can be used to assess not only the pathogens tiled on the nucleic acid array, but also those that are not tiled on the array. As appreciated by those skilled in the art, gene conservation may occur within the same species or genus, or among different species or genera. It can occur at the nucleic acid sequence level, the amino acid sequence level, or the protein three-dimensional level. For instance, the staphylococcal enterotoxin (SE) serotypes SEA, SED, and SEE are closely related by amino acid sequence, while SEB, SEC1, SEC2, SEC3, and the streptococcal pyrogenic exotoxin (SPE) share key amino acid residues with the other toxins, but exhibit only weak sequence homology overall. However, there are considerable similarities in the known three-dimensional structures of SEA, SEB, SEC1, SEC3, and toxic shock syndrome toxin-1 (TSST-1). Because of this structural similarity, polyclonal antibodies obtained from mice immunized with each SE or TSST-1 exhibit a low to high degree of cross-reaction. In the mouse, these antibody cross-reactions are sufficient to neutralize the toxicity of many other SE/TSST-1, depending upon the challenge dose. For example, immunization with a mixture of SEA, SEB, TSST-1 and SPE-A has been shown to be sufficient to provide antibody protection from a challenge with numerous other component toxins, singly or in combination. Genotyping or antigen validation of any viral or non-viral species/strains can be performed using the nucleic acid arrays of the present invention and according to the methods described herein.

The nucleic acid arrays of the present invention can also be used to identify or evaluate agents capable of inhibiting or reducing the growth or virulence of a pathogen of interest. Methods suitable for this purpose typically include the steps of (1) contacting a molecule of interest with a culture comprising the pathogen, or administrating the molecule to an animal model affected by the pathogen; and (2) hybridizing a nucleic acid sample prepared from the culture or animal model to a nucleic acid array of the present invention. Changes in the hybridization signals in the presence of the molecule of interest, as compared to control hybridization signals (e.g., hybridization signals in the absence of the molecule), can be used to determine the effect of the molecule on the growth or virulence of the pathogen. Any type of agent can be evaluated according to the present invention, such as small molecules, antibodies, peptides, or peptide mimics.

Any biological sample may be analyzed according to the present invention. Suitable biological samples include, but are not limited to, pus, blood, urine, or other body fluid, tissue or waste samples. Food, environmental, pharmaceutical or other types of samples can also be analyzed. In many embodiments, bacteria or other microbes in a sample of interest are first cultured before being analyzed by a nucleic acid array of the present invention. In many other embodiments, the original samples are directly analyzed without additional culturing.

Numerous protocols are available for performing nucleic acid array analysis. Exemplary protocols include, but are not limited to, those described in GENECHIP® EXPRESSION ANALYSIS TECHNICAL MANUAL (701021 rev. 3, Affymetrix, Inc. 2002). Nucleic acid array analysis typically involves isolation of nucleic acid from a sample of interest, followed by hybridization of the isolated nucleic acid to a nucleic acid array. The isolated nucleic acid can be RNA or DNA (e.g., genomic DNA). The isolated nucleic acid may be amplified or labeled before being hybridized to a nucleic acid array.

Various methods are available for isolating or enriching RNA. These methods include, but are not limited to, RNeasy kits (provided by QIAGEN), MasterPure kits (provided by Epicentre Technologies), and TRIZOL (provided by Gibco BRL). The RNA isolation protocols provided by Affymetrix can also be employed in the present invention. See, e.g., GENECHIP® EXPRESSION ANALYSIS TECHNICAL MANUAL (701021 rev. 3, Affymetrix, Inc. 2002).

In one example, bacterial mRNA is enriched by removing 16S and 25S rRNA. Different methods are available for eliminating or reducing the amount of rRNA in a bacterial sample. For instance, the MICROBExpress kit (Ambion, Inc.) uses oligonucleotide-attached beads to capture and remove rRNA. 16S and 25S rRNA can also be removed by enzyme digestions. In the latter method, 16S and 25S rRNA are first amplified using reverse transcriptase and specific primers to produce cDNA. The rRNA is allowed to anneal with the cDNA. The sample is then treated with RNAase H, which specifically digests RNA within an RNA:DNA hybrid.

In one embodiment, isolated mRNA is amplified before being subject to nucleic acid array analysis. Suitable mRNA amplification methods include, but are not limited to, reverse transcriptase PCR, isothermal amplification, ligase chain reaction, hexamer priming, and Qbeta replicase methods. The amplification products can be either cDNA or cRNA.

Polynucleotides for hybridization to a nucleic acid array can be labeled with one or more labeling moieties to allow for detection of hybridized polynucleotide complexes. Example labeling moieties can include compositions that are detectable by spectroscopic, photochemical, biochemical, bioelectronic, immunochemical, electrical, optical or chemical means. Example labeling moieties include radioisotopes, chemiluminescent compounds, labeled binding proteins, heavy metal atoms, spectroscopic markers, such as fluorescent markers and dyes, magnetic labels, linked enzymes, mass spectrometry tags, spin labels, electron transfer donors and acceptors, and the like. In one embodiment, the enriched bacterial mRNA is labeled with biotin. The 5′ end of the enriched bacterial mRNA is first modified by T4 polynucleotide kinase with γ-S-ATP. Biotin is then conjugated to the 5′ end of the modified mRNA using methods known in the art.

Polynucleotides can be fragmented before being labeled with detectable moieties. Exemplary methods for fragmentation include, but are not limited to, heat or ion-mediated hydrolysis.

Hybridization reactions can be performed in absolute or differential hybridization formats. In the absolute hybridization format, polynucleotides derived from one sample are hybridized to the probes in a nucleic acid array. Signals detected after the formation of hybridization complexes correlate to the polynucleotide levels in the sample. In the differential hybridization format, polynucleotides derived from two samples are labeled with different labeling moieties. A mixture of these differently labeled polynucleotides is added to a nucleic acid array. The nucleic acid array is then examined under conditions in which the emissions from the two different labels are individually detectable. In one embodiment, the fluorophores Cy3 and Cy5 (Amersham Pharmacia Biotech, Piscataway, N.J.) are used as the labeling moieties for the differential hybridization format.

Signals gathered from nucleic acid arrays can be analyzed using commercially available software, such as those provide by Affymetrix or Agilent Technologies. Controls, such as for scan sensitivity, probe labeling and cDNA or cRNA quantitation, may be included in the hybridization experiments. The array hybridization signals can be scaled or normalized before being subject to further analysis. For instance, the hybridization signal for each probe can be normalized to take into account variations in hybridization intensities when more than one array is used under similar test conditions. Signals for individual polynucleotide complex hybridization can also be normalized using the intensities derived from internal normalization controls contained on each array. In addition, genes with relatively consistent expression levels across the samples can be used to normalize the expression levels of other genes.

In one embodiment, a nucleic acid array of the present invention utilizes sequences generated from multiple complete genomes per species, thereby ensuring substantial coverage of all identifiable ORFs. In another embodiment, the parent sequences employed are derived from the highly conserved regions of each ORF. As a consequence, strains not included in the array design have a higher probability of being recognized than if individual sequences were used.

Probes for the intergenic sequences can also be included in a nucleic acid array of the present invention. These probes allow for the detection of unidentified ORFs or other expressible sequences. These intergenic probes are also useful for mapping transcription factor binding sites, identifying operons, or determining promoters, termination sites or other cis-acting regulatory elements.

The present invention also features protein arrays for the concurrent or discriminable detection of multiple strains of different non-viral or viral species. Each protein array of the present invention includes probes which can specifically bind to protein products of different non-viral or viral species. In one embodiment, the probes on a protein array of the present invention are antibodies. Many of these antibodies can bind to the respective proteins with an affinity constant of at least 10⁴ M⁻¹, 10⁵ M⁻¹, 10⁶ M⁻¹, 10⁷ M⁻¹, or stronger. In many instances, an antibody for a specified protein does not bind to other proteins expressed in the strains being analyzed. Suitable antibodies for the present invention include, but are not limited to, polyclonal antibodies, monoclonal antibodies, chimeric antibodies, single chain antibodies, synthetic antibodies, Fab fragments, or fragments produced by a Fab expression library. Other peptides, scaffolds, antibody mimics, high-affinity binders, or protein-binding ligands can also be used to construct the protein arrays of the present invention.

Numerous methods are available for immobilizing antibodies or other probes on a protein array of the present invention. Examples of these methods include, but are not limited to, diffusion (e.g., agarose or polyacrylamide gel), surface absorption (e.g., nitrocellulose or PVDF), covalent binding (e.g., silanes or aldehyde), or non-covalent affinity binding (e.g., biotin-streptavidin). Examples of protein array fabrication methods include, but are not limited to, ink-jetting, robotic contact printing, photolithography, or piezoelectric spotting. The method described in MacBeath and Schreiber, SCIENCE, 289: 1760-1763 (2000) can also be used. Suitable substrate supports for a protein array include, but are not limited to, glass, membranes, mass spectrometer plates, microtiter wells, silica, or beads.

The protein-coding sequence of a gene can be determined by a variety of methods. For instance, many protein sequences can be obtained from NCBI or other public or commercial sequence databases. Protein-coding sequences can also be extracted from non-intergenic parent sequences by using an open reading frame (ORF) prediction program. Non-limiting examples of ORF prediction programs include GeneMark (provided by the European Bioinformatics Institute), Glimmer (provided by TIGR), and ORF Finder (provided by NCBI).

In one embodiment, a protein array of the present invention includes at least 2, 5, 10, 20, 30, 40, 50, 100, 200, 300, 400, 500, 1,000, 2,000, 3,000, 4,000, 5,000 or more probes, each of which can specifically bind to a protein encoded by a different respective non-intergenic sequence selected from SEQ ID NOs: 1-18,598, or by the gene that corresponds to the non-intergenic sequence.

In another embodiment, a protein array of the present invention comprises (1) a first plurality of probes, each of which is specific to a different respective strain selected from a first species, and (2) a second plurality of probes, each of which is specific to a different respective strain selected from a second species. In many examples, the protein array further includes a third plurality of probes, each of which is specific to a different respective strain selected from a third species. The first, second, or third species can be, for example, Streptococcus pyogenes, Streptococcus agalactiae, or Staphylococcus epidermidis. Non-limiting examples of strains of these species include SSI-1, 2F3, Manfredo, MGAS315, MGAS8232, or SF370 for Streptococcus pyogenes; 2603, A909, or NEM316 for Streptococcus agalactiae; and ATCC12228, ATCC14990, O-47, RP62A, or SR1 for Staphylococcus epidermidis.

A protein array of the present invention can also include probes that are common to two or more strains of the same species. As used herein, a probe on a protein array is “specific” to a strain selected from a group if the probe can bind to a protein of that strain, but not to proteins of other strains in the group. Where a probe on a protein array can bind to proteins from two or more strains, the probe is said to be “common” to these strains.

The present invention also features polynucleotide collections. Each polynucleotide in a collection of the present invention is capable of hybridizing under stringent or nucleic acid array hybridization conditions to a sequence selected from SEQ ID NOs: 1 to 18,598, or the complement thereof. In one embodiment, the collection includes at least 2, 3, 4, 5, 10, 20, 30, 40, 50, 100, 500, 1,000 or more different polynucleotides, each of which is capable of hybridizing under stringent or nucleic acid array hybridization conditions to a different respective sequence selected from SEQ ID NOs: 1 to 18,598, or the complement thereof. In another embodiment, the collection includes at least 1, 2, 3, 4, 5, 10, 20, 30, 40, 50, 100, 500, 1,000 or more parent sequences depicted in SEQ ID NOs: 1 to 18,598, or the complement(s) thereof. The present invention contemplates any combination of SEQ ID NOs: 1 to 18,598, including but not limited to, any combination of SEQ ID NOs: 1-5,840, of SEQ ID NOs: 5,841-10,822, of SEQ ID NOs: 10,823-18,217, or of SEQ ID NOs: 18,218-18,598.

In one embodiment, a polynucleotide collection of the present invention includes at least 1, 2, 3, 4, 5, 10, 20, 30, 40, 50, 100, 500, 1,000 or more oligonucleotide probes depicted in SEQ ID NOs: 18,599 to 605,357. In another embodiment, a polynucleotide collection of the present invention includes all of the probes depicted in SEQ ID NOs: 18,599 to 605,357.

In addition, the present invention features collections of polypeptides encoded by the non-intergenic sequences selected from SEQ ID NOs: 1 to 18,598 or their corresponding genes. Polypeptides encoded by any combination of SEQ ID NOs: 1 to 18,598 or their corresponding genes are contemplated by the present invention. The present invention also features kits including at least 1, 2, 3, 4, 5, 10, 20, 30, 40, 50, 100, 500, 1,000 or more polynucleotides or polypeptides of the present invention.

It should be understood that the above-described embodiments and the following examples are given by way of illustration, not limitation. Various changes and modifications within the scope of the present invention will become apparent to those skilled in the art from the present description.

E. Examples

### EXAMPLE 1

### Nucleic Acid Array

The parent sequences depicted in SEQ ID NOs: 1-18,598 and/or their sequence segments were submitted to Affymetrix for custom array design. Probes with 25 non-ambiguous bases were selected. The final set of selected probes is depicted in SEQ ID NOs: 605,358 to 1,276,209. The specificity of each probe to different strains of Streptococcus pyogenes, Streptococcus agalactiae or Staphylococcus epidermidis is also indicated in SEQ ID NOs: 605,358 to 1,276,209 (see supra).

The perfect mismatch probe for each probe in SEQ ID NOs: 605,358 to 1,276,209 was also prepared. A perfect mismatch probe is identical to the corresponding perfect match probe except at position 13 where a single-base substitution was made. The substitutions were A to T, T to A, G to C, or C to G. The final array contains 673,599 perfect match probes and 673,599 mismatch probes, which include 10,761 Streptococcus probe sets, 7,740 Staphylococcus probe sets, and a number of exogenous control probe sets.

Affymetrix's strategy for nucleic acid array design can be found in THE GENECHIP® SYSTEM—AN INTEGRATED SOLUTION FOR EXPRESSION AND DNA ANALYSIS (Part Number 701307 Rev1, Affymetrix, Inc. 2003), which is incorporated herein by reference in its entirety. Strategies for manufacturing and using nucleic acid arrays can also be found in U.S. Pat. Nos. 5,445,934; 5,744,305; 5,945,334; 6,040,138; 6,261,776; 6,291,183; 6,346,413; and 6,399,365, all of which are incorporated herein by reference.

### EXAMPLE 2

### Analysis of the Accuracy of the Nucleic Acid Array of Example 1

An analysis can be conducted to confirm the performance of the nucleic acid array of Example 1 with respect to sequenced Streptococcus pyogenes, Streptococcus agalactiae and Staphylococcus epidermidis genomes. Each parent sequence in SEQ ID NOs: 1-18,217 is derived from the transcript(s) or intergenic sequence(s) of one or more Streptococcus pyogenes, Streptococcus agalactiae or Staphylococcus epidermidis strains. If at least 70% of the oligonucleotide probes for a parent sequence are present in the genome of a Streptococcus pyogenes, Streptococcus agalactiae or Staphylococcus epidermidis strain, then the parent sequence is theoretically predicted to be “present” in the genome of that strain. In some cases, present calls can be made on the basis of 100% of the probes being present. The theoretical predictions are compared to the actual results of DNA hybridization experiments using the nucleic acid array of Example 1 to determine the hybridization accuracy of the custom-made array.

### EXAMPLE 3

### Sample Preparation for Monitoring Gene Expression

Total RNA of Streptococcus pyogenes, Streptococcus agalactiae or Staphylococcus epidermidis strain(s) is isolated under a control condition or a test condition. Under the test condition, bacterial cells are either differentially treated or have a divergent genotype. cDNA is synthesized from total RNA of the control or test sample as follows. 10 μg total RNA is incubated at 70° C. with 25 ng/μl random hexamer primers for 10 min followed by 25° C. for 10 min. Mixtures are then chilled on ice. Next, 1×cDNA buffer (Invitrogen), 10 mM DTT, 0.5 mM dNTP, 0.5 U/μl SUPERase-In (Ambion), and 25 U/μl SuperScript II (Invitrogen) are added. For cDNA synthesis, mixtures are incubated at 25° C. for 10 min, then 37° C. for 60 min, and finally 42° C. for 60 min. Reactions are terminated by incubating at 70° C. for 10 min and are chilled on ice. RNA is then chemically digested by adding 1N NaOH and incubation at 65° C. for 30 min. Digestion is terminated by the addition of 1N HCl. cDNA products are purified using the QIAquick PCR Purification Kit in accordance with the manufacturer's instructions. Next, 5 μg of cDNA product is fragmented by first adding 1×One-Phor-All buffer (Amersham Pharmacia Biotech) and 3U DNase I (Amersham Pharmacia Biotech) and then incubating at 37° C. for 10 min. DNase I is then inactivated by incubation at 98° C. for 10 min. Fragmented cDNA is then added to 1×Enzo reaction buffer (Affymetrix), 1×CoCl₂, Biotin-ddUTP and 1×Terminal Transferase (Affymetrix). The final concentration of each component is selected according to the manufacturer's recommendations. Mixtures are incubated at 37° C. for 60 min and then stopped by adding 2 μl of 0.5 M EDTA. Labeled fragmented cDNA is then quantitated spectrophotometrically and 1.5 μg labeled material is hybridized to a nucleic acid array of the present invention at 45° C. for 15 hr.

mRNA or cRNA prepared from Streptococcus pyogenes, Streptococcus agalactiae and Staphylococcus epidermidis strain(s) can also be used for nucleic acid hybridization. mRNA or cRNA can be enriched, fragmented, and labeled according to the procedures described in GENECHIP® EXPRESSION ANALYSIS TECHNICAL MANUAL (701021 rev. 3, Affymetrix, Inc. 2002), which is incorporated herein by reference in its entirety.

### EXAMPLE 4

### Sample Preparation for Genotyping

Streptococcus pyogenes, Streptococcus agalactiae or Staphylococcus epidermidis strains are grown overnight in a 2-ml culture. Cells are harvested and lysed in a Bio101 FastPrep bead-beater (2×20s cycles). Chromosomal DNA is prepared using the Qiagen DNeasy Tissue kit following the manufacturer's instructions. Approximately 10 μg of DNA is made up to a 60 μl volume in nuclease free water. 20 μl 1N NaOH is added to remove residual RNA and the mixture is incubated at 65° C. for 30 min. 20 μl of 1N HCl is added to neutralize the reaction. The DNA is concentrated by ethanol precipitation using ammonium acetate and re-suspended in a 47 μl volume followed by a 5 min boiling step to denature the double-stranded DNA. The DNA is quantified by reading the absorbance at 260 nm. 40 μl of DNA is fragmented by treatment with DNase (0.6 U/μg DNA) in the presence of 1×One-Phor-All buffer (Amersham Pharmacia) in a total volume of 50 μl for 10 min at 37° C. followed by a 10 min incubation at 98° C. to inactivate the enzyme. 39 μl of fragmented DNA is end-labeled with biotin using the Enzo Bioarray Terminal Labeling kit (Affymetrix). 1.5 μg of labeled DNA is hybridized overnight to a nucleic acid array of the present invention in a mixture containing Oligo B2 (Affymetrix), herring sperm DNA, BSA and a standard curve reagent.

### EXAMPLE 5

### Genotyping Using the Nucleic Acid Array of Example 1

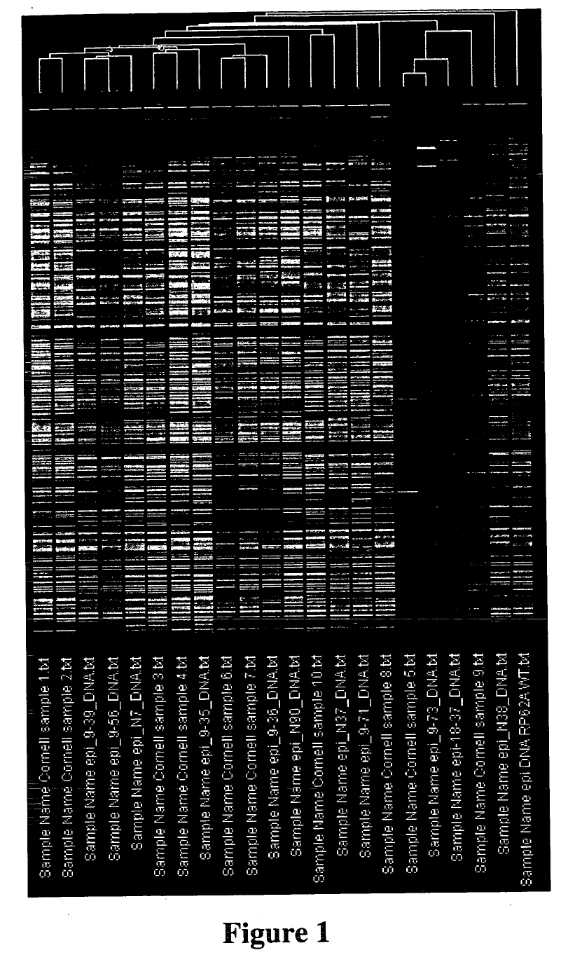

FIG. 1 represents a hierarchical clustering of 21 strains purported to be Staphylococcus epidermidis. All strains were obtained from the pediatric intensive care units or from normal neonates or nurses at two major New York hospitals. Each column represents a strain and each of 7,810 rows represents a qualifier derived from the Staphylococcus epidermidis and Staphylococcus aureus parent sequence sets. Color represents the intensity of the hybridization signal on the nucleic acid array compared to the control strain, Staphylococcus aureus RP62A (in the last column on the right). Signal intensity increases from blue to yellow to red. The tree illustrates the utility of the array in identifying epidemiological relationships between strains in a hospital outbreak. Four strains (5, 9-73, 18-37, and 9) gave very low signal on this array. Three of them have been re-typed by standard microbiological methods and shown to be either Staphylococcus aureus or non-Staphylococcus epidermidis coagulase-negative isolates.

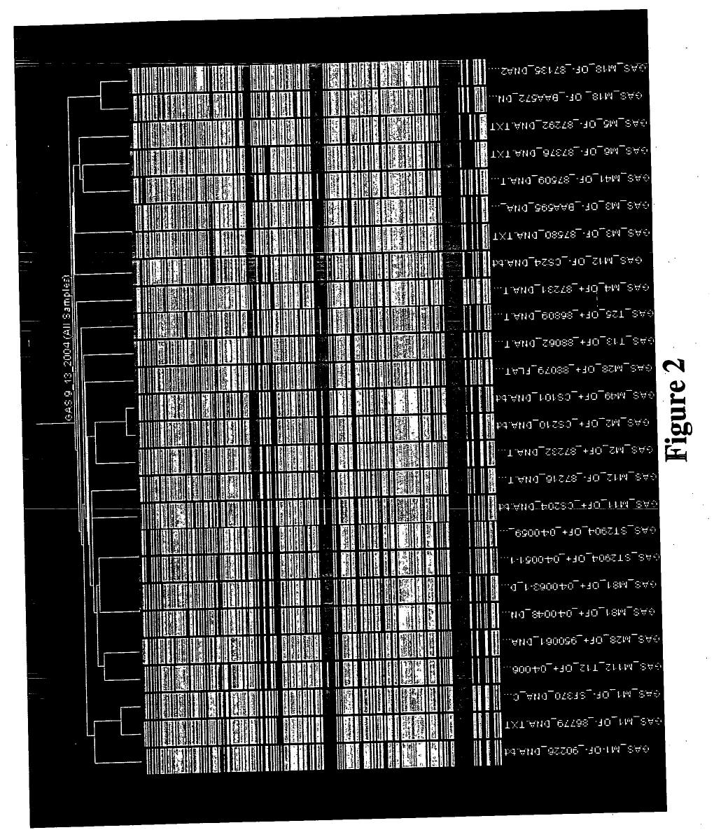

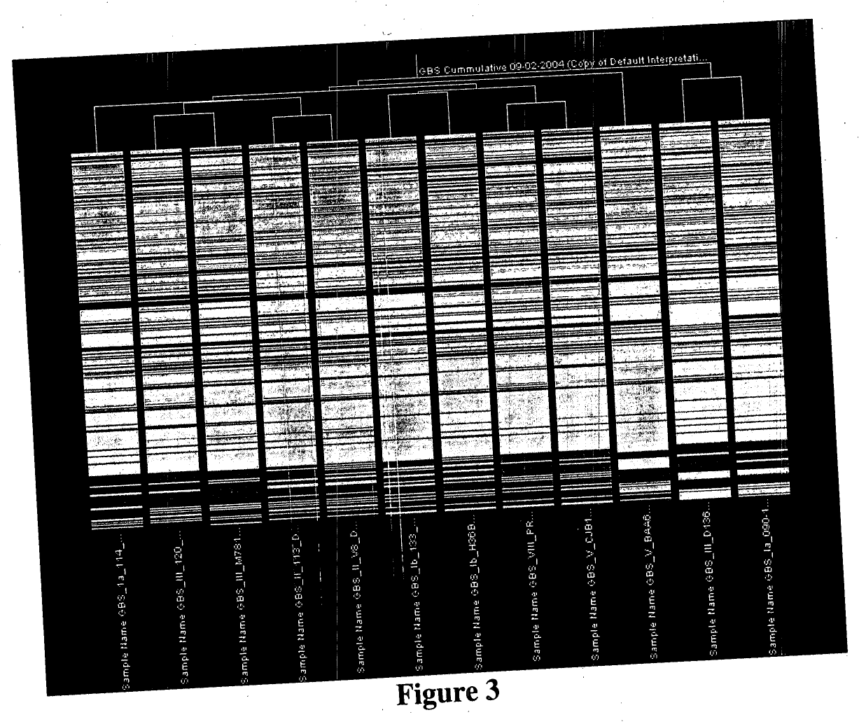

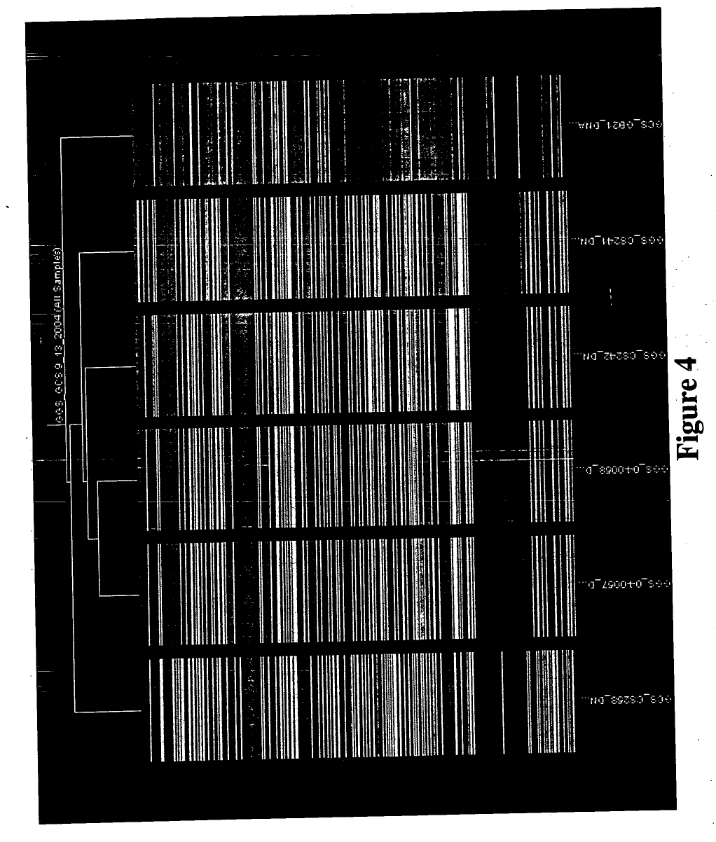

FIGS. 2, 3, and 4 depict the dendrograms of Group A streptococci (GAS), Group B streptococci (GBS), and Group C/G streptococci (GCS/GGS), respectively. Like the purported Staphylococcus epidermidis strains in FIG. 1, the strains in FIGS. 2-4 were all clinical isolates at one time, and came from different geographical settings and reflected a spread of diverse serotypes. Colors go from blue (absent) to green (present). The dendrogram of the GCS/GGS strains, as shown in FIG. 4, is mostly blue because of the stringency at which the nucleic acid array was designed. However, FIG. 4 illustrates the similarity among the hybridization patterns of different GCS/GGS strains.

### EXAMPLE 6

### Genetic Characterization of Disease-Associated Staphylococcus epidermidis Isolates

Staphylococcus epidermidis is a normal inhabitant of the skin and mucosal surfaces of healthy individuals. The organism is also a major cause of nosocomial sepsis, particularly in neonates and immunocompromised patients with indwelling devices. Studies have indicated that there is a correlation between the ability of S. epidermidis to form a biofilm and its ability to cause infection. It is thought that products of the intercellular adhesion (ica) locus provide the organism with the capability to form a biofilm on implanted medical devices, which in turn provides a site for multiplication and subsequent dissemination to other sites. However, recent reports have shown that both biofilm-forming and -deficient strains are pathogenic in mouse models of infection, suggesting that additional virulence factors contribute to the pathogen's ability to modulate disease.

In this Example, the nucleic acid array of Example 1 was used to study the genetic composition of 11 S. epidermidis strains isolated from the blood of premature neonates with sepsis and 7 skin-isolates from healthy term neonates or health-care workers. The results of this study indicate that (1) disease-associated strains are highly related to one another but are divergent from skin isolates from healthy neonates and healthcare workers; (2) most known virulence factors are present in nearly all of these strains; and (3) 30 genes, including several potential virulence factors and several conserved hypothetical proteins, are unique to the infection-associated strains.

i). Microarray Design

As described above, the design of the nucleic acid array of Example 1 was partially based on the sequences of two complete genomes, ATCC12228 (Zhang, et al., MOLEC. MICROBIOL., 49:1577-1593 (2003)) and RP62A (TIGR), the unfinished genomes of three other strains, 047 (Incyte, Wilmington, Del.), SR1 (GlaxoSmithKline, Philadelphia, Pa.) and ATCC14490 (Genome Therapeutics, Waltham, Mass.), and on individual records in GenBank. ORFs were obtained from the published sets for the complete genomes, and from GenBank CDS annotation. Glimmer 2.02 was used for ORF prediction for unannotated records and unfinished genomes. Intergenic regions greater than 50 nt in length were collected from ATCC12228 and RP62A, based on the published ORF coordinates. Highly repetitive, variable sequences such as those found in surface proteins, were deleted from the sequences prior to clustering in order to force the common regions of these genes into alignments. ORFs and intergenic sequences were separately clustered using CAT4.5 software (Doubletwist) to generate consensus sequences. Orthologs that did not meet the clustering thresholds of 97% identity over 60 nt formed separate consensus sequences which were tiled independently. The final design contained 4,449 S. epidermidis ORFs, 2,871 S. epidermidis intergenic regions (both strands), and 40 S. epidermidis tRNA and rRNA sequences. In addition, 380 S. aureus consensus sequences, mainly virulence and antibiotic resistance genes, were taken from U.S. patent application Ser. No. 10/859,198 entitled “Nucleic Acid Arrays for Detecting Multiple Strains of A Non-Viral Species” and filed Jun. 3, 2004 (by William M. Mounts et al.). For most (89%) of the qualifiers, 39 probe pairs were tiled.

ii). Strains Used in this Study

Strains of S. epidermidis were obtained from infants and health care workers at two New York City teaching hospitals. Infant samples included those from neonates with infections and from the skin of healthy babies. Table 2 depicts the S. epidermidis isolates used in the study. The isolates from healthy samples are also referred to as commensal isolates.

TABLE 2
S. epidermidis Strains

| Isolate # | Donor | Site | Clinical History |
|---|---|---|---|
| 3 | Baby 1 | blood | sepsis |
| 4 | Baby 1 | Line | |
| | | (central venous catheter) | |
| 1 | Baby 3 | blood | sepsis |
| 2 | Baby 3 | blood | |
| 6 | Baby 3 | blood | |
| 7 | Baby 3 | line | |
| | | (central venous catheter) | |
| 8 | Baby 3 | skin | |
| 9 | Baby 6 | skin | healthy |
| 10 | Baby 7 | skin | healthy |
| 9-35 | Baby 35 | blood | sepsis |
| 9-36 | Baby 36 | blood | sepsis |
| 9-39 | Baby 39 | eye | conjunctivitis |
| 9-56 | Baby 56 | blood | sepsis |
| 9-71 | Baby 71 | blood | sepsis |
| N7 | Nurse 7 | skin | healthy |
| N37 | Nurse 37 | skin | healthy |
| N38 | Nurse 38 | skin | healthy |
| N90 | Nurse 90 | skin | healthy |

iii). Methods

DNA was prepared for hybridization as described in Dunman, et al., J. CLIN. MICROBIOL., 42:4275-4283 (2004). Cells from 1.5 ml of an overnight culture were lysed, and DNA (chromosomal and plasmid) was purified on a Qiagen DNA tissue easy column. Prior to labeling, 2 μg of each DNA preparation was subjected to electrophoresis on a 0.8% agarose gel to assess integrity. DNA (5 μg) was denatured at 90° C. for 3 minutes, rapidly cooled, and fragmented and labeled according to the Affymetrix protocols for labeling mRNA for antisense prokaryotic arrays. A 1.5 μg aliquot was hybridized to the nucleic acid array of Example 1 and processed according to the Affymetrix protocols. Signal intensities were floored by raising any raw values less than 0.01 to 0.01, then normalized to account for loading errors and differences in labeling efficiencies by dividing each signal by the median signal intensity for each individual chip. Affymetrix Present/Absent calls were not used since it has been shown that they are inaccurate (many false positive errors) for DNA hybridization. See, for example, Dunman, et al., supra. Instead, genes were considered to be Present if their normalized signal was equal to or greater than 0.475, as described below. Data were analyzed using GeneSpring version 6.2 and Spotfire version 7.3.

iv). Adjustment of Present/Absent Calls

It has been shown that the Affymetrix Present/Absent calls, when used with DNA hybridization protocols, include many false-positive errors. Therefore, a method was developed for determining the presence or absence of each gene based on the signals obtained with strains for which a complete genome sequence is available and for which the presence or absence of each qualifier on the array can therefore be predicted. See, for example, Dunman, et al., supra. The same method was employed for the nucleic acid array of Example 1, using strain RP62A.

For each qualifier, each perfect-match oligonucleotide was searched in the genome of RP62A, and the qualifier was predicted to be called Present if at least 70% of the oligonucleotides were contained within the genome. An adjusted present call cutoff value was then set such that 90% of the qualifiers known to be present in RP62A would have signals greater than this value. A second cutoff was defined such that 90% of the qualifiers expected to be Absent would have signal intensities below this second value. Qualifiers with values between the two cutoffs are considered indeterminable. This method does not increase the total number of correctly called qualifiers compared to the default GCOS algorithm, but it is more conservative, generating fewer false-positive calls and classifying more qualifiers as indeterminable (equivalent in principle to the Affymetrix “marginal” call). In addition, this method allows calls to be made based on a chip-normalized rather than the raw signal values. This provides a value that can then be used to make Present/Absent calls for strains whose sequences are not known.

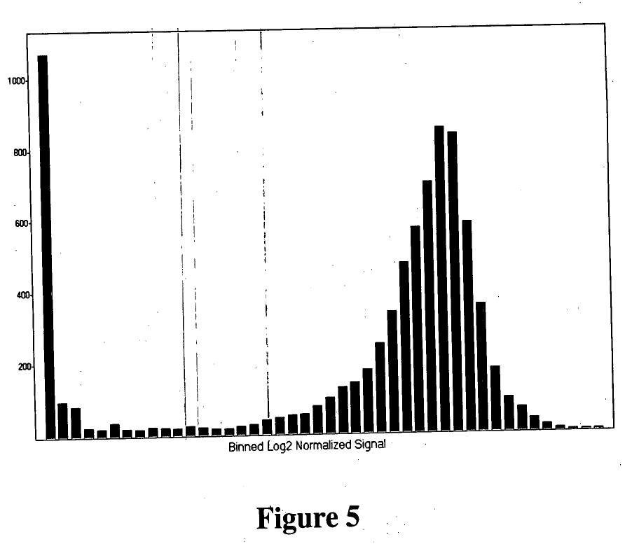

The distribution of expected present and absent qualifiers for RP62A is shown in FIG. 5. Blue indicates predicted absent, and red represents predicted present (based on presence of at least 70% of perfect-match oligonucleotides).

A summary of the number of predicted Present calls and those produced by the Affymetrix GCOS software are shown in Table 3.

TABLE 3
Predicted vs Affymetrix GCOS Present Calls for RP62A Control Strain

| | Affymetrix GCOS | Adjusted Cutoffs |
|---|---|---|
| Predicted Present | 6,083 | 6,083 |
| Predicted Absent | 1,657 | 1,657 |
| Called Present, total | 6,409 | 5,561 |
| Called Present, correct | 6,059 | 5,474 |
| Called Present, incorrect | 350 | 87 |
| Called Absent, total | 1,305 | 1,665 |
| Called Absent, correct | 1,290 | 1,493 |
| Called Absent, incorrect | 15 | 165 |
| Indeterminable/Marginal | 26 | 514 (74 predicted A, |
| | | 440 predicted P) |

Adjusted normalized cutoff values were: Present, ≧0.475; Absent, ≦0.205, Indeterminable 0.205-0.405. Numbers are the number of qualifiers.

v). Confirmation of Selected Present/Absent Calls

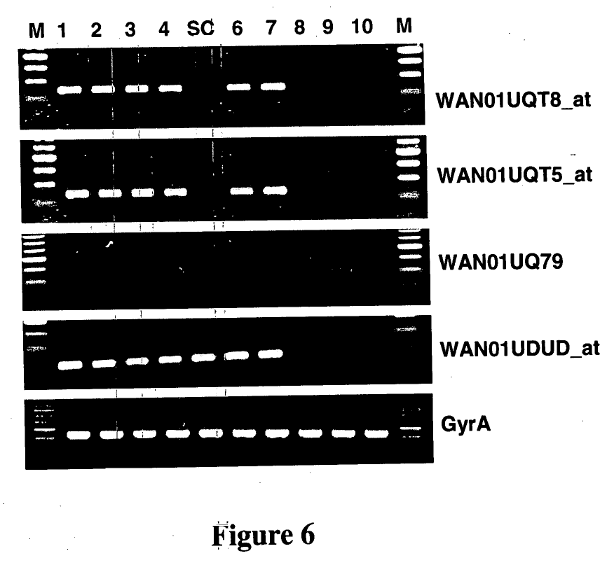

Several genes were selected for confirmation of the nucleic acid array results by PCR (FIG. 6, Table 4). gyrA was used as a positive control. All PCR results were as expected from the nucleic acid results. FIG. 6 shows PCR Amplification of Selected Genes. PCR amplification was performed from strains 1-10. M represents markers; and SC indicates control S. caprae strain.

TABLE 4
PCR Confirmation of Selected Genes

| | | Strain | | | | | | | | |
|---|---|---|---|---|---|---|---|---|---|---|
| Qualifier | Gene | 1 | 2 | 3 | 4 | 6 | 7 | 8 | 9 | 10 |
| WAN01UQT8_at | | + | + | + | + | + | + | − | − | − |
| WAN01UQT5_at | | + | + | + | + | + | + | − | − | − |
| WAN01UQ79_at | sdrF | + | + | + | + | + | + | − | + | + |
| WAN01UDUD_at | mecI | + | + | + | + | + | + | − | − | − |
| WAN01UO6M_at | gyrA | + | + | + | + | + | + | + | + | + |

vi). Use of Nucleic Acid Array to Monitor Strain Relatedness

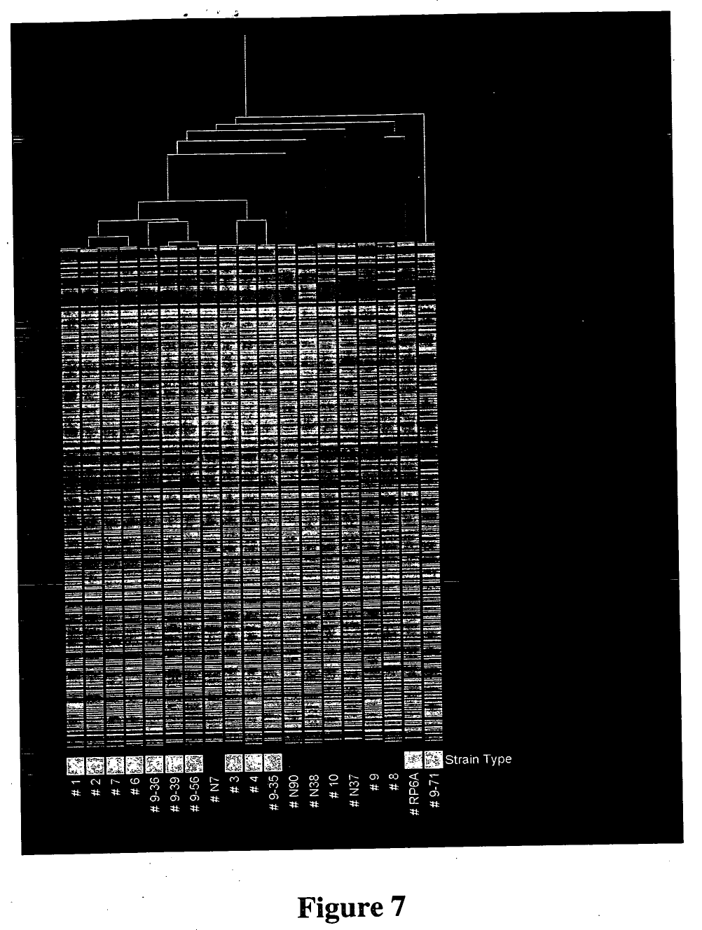

Hierarchical clustering was used to develop a dendrogram comparing the normalized signal intensity of each qualifier for any strain to the intensity of the same qualifier across all strains. Strains that have similar patterns of signal intensities are positioned closer together on the dendrogram than strains with divergent genomic composition. FIG. 7 shows the dendrogram and heat map resulting from analysis of the S. epidermidis strains described in Table 2. It is evident that the strains derived from neonatal infections are, in general, more closely related to one another than to the strains obtained from normal neonates and from health care workers, with the exception of strain 9-71, which is an outlier compared to all other strains. Multiple isolates from the same infant (strains 3 & 4, and 1,2,6 & 7) are the most closely related. Most of the strains from healthy donors, in addition to being distinguishable from those causing infections, also show considerably more diversity. Of interest is the fact that strain 8, obtained one month later as a skin isolate, is unrelated to earlier isolates from this baby's infection.

Clustering was performed in GeneSpring on normalized signal intensities, using standard correlation as the similarity measure and using data from all qualifiers (intergenics, ORFS and RNA). For this and all subsequent heat map figures, each column represents a strain; and each row signifies a qualifier. Genes are colored by normalized signal intensity, red indicating high values, blue low values, yellow average values. The bottom bar indicates strain type: yellow indicates infection-related; red indicates skin isolates. The RP62A control strain is coded turquoise.

vii). Analysis of Virulence Factors, Antibiotic Resistance Genes, and Regulatory Regions

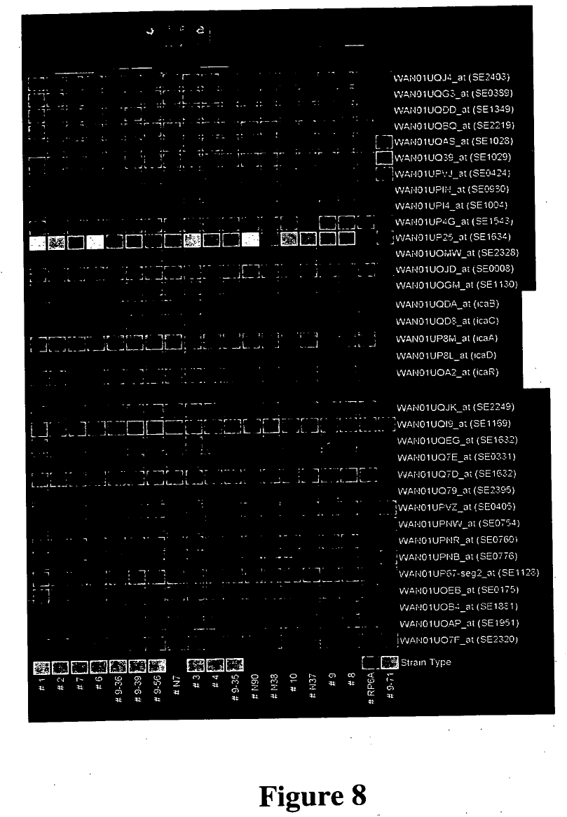

Nucleic acid array analysis provides the ability to simultaneously monitor the presence or absence of more than 4,000 genes in each strain. Table 5 lists characterized virulence genes adapted from the publication describing the ATCC12228 genome sequence (Zhang, et al., supra). Table 5 also includes the ica gene cluster, which is not present in this strain. FIG. 8 shows the presence or absence of the genes in Table 5 in the clinical isolates. Two strains, 9 and 9-71, are missing the entire ica operon, icaABCDR. Two strains, 8 and the control RP62A, lack sdrF (bone sialoprotein, SE2395). Strains 8 and 9-71 lack accumulation-associated protein (SE0175). 9-71 also lacks SE0776 (67 kDa myosin-crossreactive streptococcal antigen-like protein). The remainder of these virulence genes is present in all strains examined.

TABLE 5
Putative Virulence Factors in S. epidermidis ATCC12228

| | | | Gene | |
|---|---|---|---|---|
| Qualifier | Protein | ORF | name | Function |
| WAN01UO7F_at | Lactococcal lipoprotein | SE2320 | | Possible host cell attachment |
| WAN01UOAP_at | Putative protein similar to | SE1951 | | Possible host cell attachment |
| | attachment and virulence | | | |
| WAN01UOB4_at | Autolysin | SE1881 | atlE | Adhere to polymers |
| WAN01UOEB_at | Cell accumulation | SE0175 | aap | Accumulation associated |
| | | | | protein |
| WAN01UOGM_at | Putative 5′-3′ exonuclease | SE1130 | | Possibly degrades host |
| | | | | nucleic acid |
| WAN01UOJD_at | Beta-haemolysin | SE0008 | | Phospholipase C synthesis |
| WAN01UOMW_at | Putative carboxyl esterase | SE2328 | | Possibly degrade lipids |
| WAN01UP25_at | Delta-haemolysin | SE1634 | hld | Destruction of blood and |
| | | | | tissue cells |
| WAN01UP4G_at | Serine protease V8 protease | SE1543 | sspA | Degrade or digest proteins |
| WAN01UP6C_at | Extracellular matrix binding | SE1128 | embp | Fibronectin binding protein |
| | protein Embp | | | |
| WAN01UPI4_at | Thermonuclease | SE1004 | nuc | Digest host nucleic acids |
| WAN01UPIN_at | Lysophospholipase | SE0980 | | Possibly degrades lipids |
| WAN01UPNB_at | 67 KDa myosin cross-reactive | SE0776 | | Cross-reactive with host |
| | protein | | | cardiac myosin |
| WAN01UPNR_at | Chitinase B | SE0760 | iraE | Invasion of skin |
| WAN01UPNW_at | Fmt | SE0754 | fmt | Autolysis and penicillin |
| | | | | resistance |
| WAN01UPVJ_at | Lipase A | SE0424 | | Possibly degrades lipids |
| WAN01UPVZ_at | Putative lipoprotein similar to | SE0405 | | Putative adhesin |
| | streptococcal PsaA | | | |
| WAN01UQ39_at | Exonuclease | SE1029 | | Possibly degrades host |
| | | | | nucleic acid |
| WAN01UQ6V_at | Similar to SE0331 (sdrG) in | | | |
| | strain SR1 | | | |
| WAN01UQ79_at | SD-rich cell surface adhesin | SE2395 | sdrF | Unknown |
| WAN01UQ7E_at | Fibrinogen binding protein | SE0331 | sdrG(fbe) | FIbrinogen binding protein |
| WAN01UQJK_at | Similar to streptococcal | SE2249 | | Unknown |
| | haemagglutinin | | | |
| WAN01UQAS_at | Exonuclease | SE1028 | | Possibly degrades host |
| | | | | nucleic acid |
| WAN01UQBQ_at | Metalloprotease | SE2219 | SepP1 | Elastase |
| WAN01UQDD_at | Protease ClpX | SE1349 | clpX | Degrade or digest proteins |
| WAN01UQEG_at | SD-rich cell surface adhesin | SE1632 | sdrF | Unknown |
| WAN01UQG3_at | Putative esterase lipase-1 | SE0389 | | Possibly digest lipids |
| WAN01UQI9_at | Elastin-binding protein | SE1169 | ebpS | Adhesion on host proteins |
| WAN01UQJ4_at | Glycerol ester hydrolase | SE2403 | geh | Degrade lipids |
| WAN01UP8L_at | Intercellular adhesion operon | | icaA | Biofilm formation |
| WAN01UQDA_at | Intercellular adhesion operon | | icaB | Biofilm formation |
| WAN01UQD8_at | Intercellular adhesion operon | | icaC | Biofilm formation |
| WAN01UP8M_at | Intercellular adhesion operon | | icaD | Biofilm formation |
| WAN01UOA2_at | Intercellular adhesion operon | | icaR | Biofilm formation |
| | regulator | | | |

Table 6 lists S. epidermidis antibiotic resistance determinants that are represented on the nucleic acid array of Example 1, and indicates whether these antibiotic resistance genes are predicted to be present or absent in each isolate. All isolates harbor genes conferring chloramphenicol (yjhl), bicyclomycin (IcaB), and cadmium resistance (cadD). Likewise, all strains carry the methicillin resistance gene mecA, but other components of the staphylococcal cassette chromosome mec (SCCmec) differ among strains. Additional differences were observed for genes typically located on the S. aureus vancomycin resistance plasmid pLWO43 as well as other resistance determinants, such as trimethoprin resistance. Collectively, these results suggest that the isolates are not clonal and that the nucleic acid array employed can provide a tool to track the transmission of resistance genes. No significant differences were observed for antimicrobial resistance determinants between infection-associated and commensal isolates.

TABLE 6
S. epidermidis Antimicrobial Resistance Determinants

| | | | Infection Associated Isolates | | | | | | | | | | | Skin Isolates | | | | | | |
|---|---|---|---|---|---|---|---|---|---|---|---|---|---|---|---|---|---|---|---|---|
| Identifier | Infection | Skin | 1 | 2 | 3 | 4 | 6 | 7 | 9_35 | 9_36 | 9_39 | 9_56 | 9_71 | 10 | 8 | 9 | N90 | N37 | N38 | N7 |
| WAN01UPOT_at | 0 | 0 | − | − | − | − | − | − | − | M | − | − | − | − | − | − | − | − | − | − |
| WAN01UG5S_at | 1 | 3 | − | − | − | − | − | − | − | M | − | + | − | + | + | − | + | − | − | − |
| WAN01UPMN_at | 1 | 2 | − | − | − | − | − | − | − | M | − | − | + | − | + | + | − | − | − | − |
| WAN01UPMO_a | 0 | 0 | − | − | − | − | − | − | − | M | − | − | − | − | − | − | − | − | − | − |
| WAN01UPMP_at | 10 | 5 | + | + | + | + | + | + | + | + | + | + | − | + | − | − | + | + | + | + |
| WAN01URBA_at | 0 | 0 | − | − | − | − | − | − | − | M | − | − | − | − | − | − | − | − | − | − |
| WAN01UNYB_at | 11 | 7 | + | + | + | + | + | + | + | + | + | + | | + | + | + | + | + | + | + |
| WAN01UR9H_at | 0 | 2 | − | − | − | − | − | − | − | M | − | − | − | − | − | − | − | + | + | − |
| WAN01URCS_at | 0 | 1 | − | − | − | − | − | − | − | M | − | − | − | − | − | − | + | − | − | − |
| WAN01UQA6_at | 11 | 7 | + | + | + | + | + | + | + | + | + | + | + | + | + | + | + | + | + | + |
| WAN01UR6N_at | 0 | 0 | − | − | − | − | − | − | − | M | − | − | − | − | − | − | − | − | − | − |
| WAN01UQIR_at | 11 | 7 | + | + | + | + | + | + | + | + | + | + | + | + | + | + | + | + | + | + |
| WAN01URAJ_at | 0 | 0 | − | − | − | − | − | − | − | M | − | − | − | − | − | − | − | − | − | − |
| WAN01UPHM_at | 11 | 7 | + | + | + | + | + | + | + | + | + | + | + | + | + | + | + | + | + | + |
| WAN01URCF_at | 0 | 0 | − | − | − | − | − | − | − | M | − | − | − | − | − | − | − | − | − | − |
| WAN01UQ3B_at | 11 | 7 | + | + | + | + | + | + | + | + | + | + | + | + | + | + | + | + | + | + |
| WAN01UQIP_at | 0 | 0 | − | − | − | − | − | − | − | M | − | − | − | − | − | − | − | − | − | − |
| WAN01UQIO_at | 0 | 0 | − | − | − | − | − | − | − | M | − | − | − | − | − | − | − | − | − | − |
| WAN01UESR_at | 0 | 0 | − | − | − | − | − | − | − | M | − | − | − | − | − | − | − | − | − | − |
| WAN01UDVP_at | 0 | 1 | − | − | − | − | − | − | − | M | − | − | − | − | − | − | − | + | − | − |
| WAN01UDUD_at | 10 | 3 | + | + | + | + | + | + | + | + | + | + | − | − | − | − | − | + | + | + |
| WAN01UDU4_at | 2 | 1 | M | M | + | M | M | M | + | M | M | M | − | − | − | M | − | + | M | M |
| WAN01UDUF_at | 11 | 6 | + | + | + | + | + | + | + | + | + | + | + | + | + | + | − | + | + | + |
| WAN01UPFG_at | 11 | 7 | + | + | + | + | + | + | + | + | + | + | + | + | + | + | + | + | + | + |
| WAN01UPUR_at | 0 | 0 | − | − | − | − | − | − | − | M | − | − | − | − | − | − | − | − | − | − |
| WAN01UQJL_at | 1 | 3 | − | − | − | − | − | − | − | M | − | + | − | + | + | − | + | − | − | − |
| WAN01UDYH_at | 0 | 0 | − | − | − | − | − | − | − | M | − | − | − | − | − | − | − | − | − | − |
| WAN01UDZ6_at | 0 | 0 | − | − | − | − | − | − | − | M | − | − | − | − | − | − | − | − | − | − |
| WAN01UDZB_at | 0 | 0 | − | − | − | − | − | − | − | M | − | − | − | − | − | − | − | − | − | − |
| WAN01UETU_at | 0 | 0 | − | − | − | − | − | − | − | M | − | − | − | − | − | − | − | − | − | − |
| WAN01UQ9Y_at | 0 | 0 | − | − | − | − | − | − | − | M | − | − | − | − | − | − | − | − | − | − |
| WAN01UEPQ_at | 11 | 6 | + | + | + | + | + | + | + | + | + | + | + | + | + | + | − | + | + | + |
| WAN01UQDW_at | 3 | 1 | − | − | + | + | − | − | − | + | M | M | − | M | − | − | − | + | − | M |
| WAN01UQUH_at | 0 | 0 | − | − | − | − | − | − | − | M | − | − | − | − | − | − | − | − | − | − |
| WAN01UQUI_at | 0 | 0 | − | − | − | − | − | − | − | M | − | − | − | − | − | − | − | − | − | − |
| WAN01UQUK_at | 0 | 0 | − | − | − | − | − | − | − | M | − | − | − | − | − | − | − | − | − | − |
| WAN01UQUJ_at | 0 | 0 | − | − | − | − | − | − | − | M | − | − | − | − | − | − | − | − | − | − |
| WAN01UQUG_at | 0 | 0 | − | − | − | − | − | − | − | M | − | − | − | − | − | − | − | − | − | − |
| WAN01UQUF_at | 0 | 0 | − | − | − | − | − | − | − | M | − | − | − | − | − | − | − | − | − | − |
| WAN01UQUE_at | 0 | 0 | − | − | − | − | − | − | − | M | − | − | − | − | − | − | − | − | − | − |
| WAN01UR9Y_at | 0 | 0 | − | − | − | − | − | − | − | M | − | − | − | − | − | − | − | − | − | − |

| Identifier | Gene | Locus | Strain | Description |
|---|---|---|---|---|
| WAN01UPOT_at | tetK | | S. epidermidis plasmid pSE-12228-C | tetracycline resistance |
| WAN01UG5S_at | aadD | | S. epidermidis isolate 210 | kanamycin nucleotidyltransferase |
| WAN01UPMN_at | arsB | SE0135 | S. epidermidis ATCC12228 | arsenic efflux pump |
| WAN01UPMO_a | arsB | SAR0691 | S. aureus MRSA | arsenical pump membrane protein |
| WAN01UPMP_at | arsB | | S. epidermidis ATCC14990 | arsenic efflux pump |
| WAN01URBA_at | bacA | SA0638 | S. aureus N315 | bacitracin resistance |
| WAN01UNYB_at | tcaB | SE0247 | S. epidermidis ATCC12228 | bicyclomycin resistance |
| WAN01UR9H_at | cadC | SAR0724 | S. aureus MRSA | putative cadmium efflux system accessory protein |
| WAN01URCS_at | cadC | | S. aureus plasmid pl258 | putative cadmium efflux system accessory protein |
| WAN01UQA6_at | cadD | SE1515 | S. epidermidis ATCC12228 | putative cadmium resistance transporter |
| WAN01UR6N_at | | SE0078 | S. epidermidis ATCC12228 | cadmium resistance transporter, putative |
| WAN01UQIR_at | Yfhl | SE2259 | S. epidermidis ATCC12228 | chloramphenicol resistance protein Yfhl |
| WAN01URAJ_at | | SA2203 | S. aureus N315 | drug resistance transporter, EmrB/QacA subfamily |
| WAN01UPHM_at | fmtc | SE1041 | S. epidermidis ATCC12228 | oxacillin resistance-related FmtC protein |
| WAN01URCF_at | fosB | SA2124 | S. aureus N315 | fosfomycin resistance protein |
| WAN01UQ3B_at | fosB | SE0231 | S. epidermidis ATCC12228 | fosfomycin resistance protein fofB |
| WAN01UQIP_at | kdpB(SCCmec) | SA0070 | S. aureus N315 | potassium-transporting ATPase B chain homologue |
| WAN01UQIO_at | kdpC(SCCmec) | SA0071 | S. aureus N315 | potassium-transporting ATPase C chain homologue |
| WAN01UESR_at | kdpE(SCCmec) | SA0066 | S. aureus N315 | similar to kdp operon transcriptional regutalory protein |
| WAN01UDVP_at | | SE0066 | S. epidermidis ATCC12228 | truncated transposase |
| WAN01UDUD_at | mecl | SERP2519 | S. epidermidis RP62A | methicillin resistance regulatory protein |
| WAN01UDU4_at | mecR1 | | S. aureus 85/2082 | methicillin resistance regulatory protein |
| WAN01UDUF_at | mecR1 | SERP2520 | S. epidermidis RP62A | methicillin resistance regulatory protein |
| WAN01UPFG_at | pbp2 | SE1138 | S. epidermidis ATCC12228 | penicillin-binding protein |
| WAN01UPUR_at | qacC′ | | S. epidermidis plasmid pSEepCH | quaternary ammonium compound resistance |
| WAN01UQJL_at | repB_1 | SA0027 | S. aureus N315 | truncated replication protein for plasmid SSC-mec |
| WAN01UDYH_at | | SAS0029 | S. aureus MSSA | conserved hypothetical protein, SSCmec |
| WAN01UDZ6_at | | | S. epidermidis SR1 | similar to hypothetical protein, Type-II SCCmec |
| WAN01UDZB_at | | SA0054 | S. aureus N315 | conserved hypothetical protein, SCCmec |
| WAN01UETU_at | | SA0076 | S. aureus N315 | hypothetical protein, SSCmec |
| WAN01UQ9Y_at | semB | SA2142 | S. aureus N315 | drug resistance transporter, EmrB/QacA subfamily |
| WAN01UEPQ_at | | SERP2528 | S. epidermidis RP62A | hypothetical protein, SSC-mec |
| WAN01UQDW_at | mobB | | S. epidermidis plasmid pSK639 | mobilization plasmid |
| WAN01UQUH_at | vanA | | S. aureus plasmid pLW043 | vancomycin/teicoplanin A-type resistance protein |
| WAN01UQUI_at | vanH | | S. aureus plasmid pLW043 | vancomycin resistance protein |
| WAN01UQUK_at | vanR | | S. aureus plasmid pLW043 | vancomycin response regulator |
| WAN01UQUJ_at | vanS | | S. aureus plasmid pLW043 | sensor histidine kinase |
| WAN01UQUG_at | vanX | | S. aureus plasmid pLW043 | vancomycin B-type resistance protein |
| WAN01UQUF_at | vanY | | S. aureus plasmid pLW043 | D-alanyl-D-alanine carboxypeptidase |
| WAN01UQUE_at | vanZ | | S. aureus plasmid pLW043 | vanZ protein VRA0043 |
| WAN01UR9Y_at | vatB | | S. aureus | acelyltransferase, resistance to virginiamycin A-like |
| | | | | antibiotics |

Several regulatory loci have been shown to be important for S. aureus pathogenesis. Although the regulatory effects of these loci are poorly defined in S. epidermidis, it seems that they may contribute to the organism's ability to cause disease. Table 7 compares the presence/absence of the S. epidermidis orthologs among the infection- and skin-isolates. Results indicate that all but one strain, contain the genes comprising the accessory gene regulator locus (agr) type 1; isolate 9 (skin) carries a variant agr type. In addition, most isolates contain other major virulence factor regulatory genes, such as sara, lyt, srr, trap, and sigB. As in the case of antibiotic determinants, no significant differences were observed among virulence factor regulatory genes between neonatal sepsis and commensal isolates.

viii). Identification of Genes Specific to Infection-Causing Strains

Given the lack of significant differences among putative virulence factors between the two isolate sets, it was anticipated that other previously uncharacterized factors may influence an isolate's ability to cause neonatal disease. To identify such factors, genes that were present in at least 7 of the 11 infection-related strains but absent in at least 5 of the 7 skin isolates were searched. Twenty six putative open reading frames matched these criteria (Table 8). Among the genes identified were a transcription regulator, six putative transporter genes, and a stress-response protein. In addition, putative enzymes, including a dehydrogenase and a phospholipase, were more frequently contained within infection-associated isolates, as were several plasmid genes. Potentially the most important difference between the infection-related and skin-colonizing isolates is the presence of all members of the arginine deiminase (arc) operon among the infection-related isolates and their absence among the isolates colonizing the skin. Genes of this operon include carbamate kinase, ornithine carbamoyltransferase, an arginine/ornithine antiporter, arginine deiminase, and arginine repressor.

TABLE 7
S. epidermidis Putative Virulence Factor Regulatory Elements

| | | | Infection-Associated Isolates | | | | | | | | | | | Skin Isolates | | | | | | |
|---|---|---|---|---|---|---|---|---|---|---|---|---|---|---|---|---|---|---|---|---|
| Identifier | Infection | Skin | 1 | 2 | 3 | 4 | 6 | 7 | 9_35 | 9_36 | 9_39 | 9_56 | 9_71 | 10 | 8 | 9 | N90 | N37 | N38 | N7 |
| WAN01UHXX_a | 11 | 7 | + | + | + | + | + | + | + | + | + | + | + | + | + | + | + | + | + | + |
| WAN01UDQJ_a | 10 | 7 | + | + | + | + | + | + | + | + | + | M | + | + | + | + | + | + | + | + |
| WAN01UP22_at | 11 | 7 | + | + | + | + | + | + | + | + | + | + | + | + | + | + | + | + | + | + |
| WAN01UGDA_at | | | | | | | | | | | | | | | | | | | | |
| WAN01UP23_at | 7 | 3 | + | + | + | + | + | + | M | M | M | M | + | + | + | − | M | + | M | M |
| WAN01UP24_at | 11 | 6 | + | + | + | + | + | + | + | + | + | + | + | + | + | − | + | + | + | + |
| WAN01UOJ4_a [symbol] | 0 | 0 | − | − | − | − | − | − | − | M | − | − | − | − | − | M | − | − | − | − |
| WAN01UOZZ_a | 0 | 1 | − | − | − | − | − | − | − | M | − | − | − | − | − | + | − | − | − | − |
| WAN01URDK_at | | | | | | | | | | | | | | | | | | | | |
| WAN01UPG9_a | 11 | 7 | + | + | + | + | + | + | + | + | + | + | + | + | + | + | + | + | + | + |
| WAN01UPGA_a [symbol] | 11 | 7 | + | + | + | + | + | + | + | + | + | + | + | + | + | + | + | + | + | + |
| WAN01UPDA_a [symbol] | 11 | 7 | + | + | + | + | + | + | + | + | + | + | + | + | + | + | + | + | + | + |
| WAN01UOCA_a [symbol] | 11 | 7 | + | + | + | + | + | + | + | + | + | + | + | + | + | + | + | + | + | + |
| WAN01UOUR_a [symbol] | 11 | 7 | + | + | + | + | + | + | + | + | + | + | + | + | + | + | + | + | + | + |
| WAN01UOSE_a [symbol] | 11 | 7 | + | + | + | + | + | + | + | + | + | + | + | + | + | + | + | + | + | + |
| WAN01UP94_a [symbol] | 11 | 7 | + | + | + | + | + | + | + | + | + | + | + | + | + | + | + | + | + | + |
| WAN01UO5M_a [symbol] | 11 | 7 | + | + | + | + | + | + | + | + | + | + | + | + | + | + | + | + | + | + |
| WAN01UP7I_at | 11 | 7 | + | + | + | + | + | + | + | + | + | + | + | + | + | + | + | + | + | + |
| WAN01UQDT_ [symbol] | 11 | 7 | + | + | + | + | + | + | + | + | + | + | + | + | + | + | + | + | + | + |
| WAN01UQG2_ [symbol] | 11 | 7 | + | + | + | + | + | + | + | + | + | + | + | + | + | + | + | + | + | + |
| WAN01UOZF_a | 11 | 7 | + | + | + | + | + | + | + | + | + | + | + | + | + | + | + | + | + | + |
| WAN01UOZI_at | 11 | 7 | + | + | + | + | + | + | + | + | + | + | + | + | + | + | + | + | + | + |
| WAN01UOZG_ [symbol] | 11 | 7 | + | + | + | + | + | + | + | + | + | + | + | + | + | + | + | + | + | + |
| WAN01UOZH_ [symbol] | 11 | 7 | + | + | + | + | + | + | + | + | + | + | + | + | + | + | + | + | + | + |
| WAN01UPEM_ [symbol] | 11 | 7 | + | + | + | + | + | + | + | + | + | + | + | + | + | + | + | + | + | + |
| WAN01UP5H_a | 11 | 7 | + | + | + | + | + | + | + | + | + | + | + | + | + | + | + | + | + | + |

| Identifier | Gene | Locus | Strain | Description |
|---|---|---|---|---|
| WAN01UHXX_a | agrA | SE1638 | S. epidermidis ATCC12228 | accessory gene regulator A |
| WAN01UDQJ_a | agrB | SE1635 | S. epidermidis ATCC12228 | accessory gene regulator B (conserved N-terminal domain) |
| WAN01UP22_at | agrC | SE1637 | S. epidermidis ATCC12228 | accessory gene regulator C (constant C-terminal domain) |
| WAN01UGDA_at | agrB | SE1635 | S. epidermidis ATCC12228 | accessory gene regulator B (S. epidermidis type 1) |
| WAN01UP23_at | agrC | SE1637 | S. epidermidis ATCC12228 | accessory gene regulator C (S. epidermidis type 1) |
| WAN01UP24_at | agrD | SE1636 | S. epidermidis ATCC12228 | AgrD protein S. epidermidis type 1 |
| WAN01UOJ4_a [symbol] | agrB | | S. epidermidis N10191 | accessory gene regulator protein B (S. epidermidis type strain N10191) |
| WAN01UOZZ_a | agrC | | S. epidermidis N10191 | accessory gene regulator protein C (S. epidermidis strain N10191) |
| WAN01URDK_at | agrD | | S. epidermidis N10191 | accessory gene regulator protein D (S. epidermidis strain N10191) |
| WAN01UPG9_a | arlR | SE1100 | S. epidermidis ATCC12228 | putative response regulator ArlR |
| WAN01UPGA_a [symbol] | arlS | SE1099 | S. epidermidis ATCC12228 | putative protein histidine kinase arlS |
| WAN01UPDA_a [symbol] | comGC | SE1228 | S. epidermidis ATCC12228 | exogenous DNA-binding protein comGC |
| WAN01UOCA_a [symbol] | | SE1749 | S. epidermidis ATCC12228 | lytic regulatory protein |
| WAN01UOUR_a [symbol] | lytR | SE1883 | S. epidermidis ATCC12228 | lyt divergon expression attenuator LytR |
| WAN01UOSE_a [symbol] | lytS | SE2011 | S. epidermidis ATCC12228 | two-component sensor histidine kinase |
| WAN01UP94_a [symbol] | phoP | SE1369 | S. epidermidis ATCC12228 | alkaline phosphatase synthesis transcriptional regulatory protein |
| WAN01UO5M_a [symbol] | phoR | SE1368 | S. epidermidis ATCC12228 | alkaline phosphatase synthesis sensor protein |
| WAN01UP7I_at | rot | SE1435 | S. epidermidis ATCC12228 | repressor of toxins Rot |
| WAN01UQDT_ [symbol] | | SE0478 | S. epidermidis ATCC12228 | histidine protein kinase |
| WAN01UQG2_ [symbol] | sarA | SE0390 | S. epidermidis ATCC12228 | staphytococcal accessory regulator A |
| WAN01UOZF_a | rsbU | SE1671 | S. epidermidis ATCC12228 | sigmaB regulation protein RsbU |
| WAN01UOZI_at | rsbW | SE1669 | S. epidermidis ATCC12228 | anti-sigmaB factor |
| WAN01UOZG_ [symbol] | rsbV | SE1670 | S. epidermidis ATCC12228 | anti-sigmaB factor antagonist |
| WAN01UOZH_ [symbol] | sigB | SE1668 | S. epidermidis ATCC12228 | sigma factor B |
| WAN01UPEM_ [symbol] | srrA | SE1176 | S. epidermidis ATCC12228 | staphylococcal respiratory response protein SrrA |
| WAN01UP5H_a | traP | SE1514 | S. epidermidis ATCC12228 | signal transduction protein TRAP |

TABLE 8
S. epidermidis Infection-Associated Genes

| | | | Infection Associated Isolate | | | | | | | | | | | Skin Isolate | | | | | | |
|---|---|---|---|---|---|---|---|---|---|---|---|---|---|---|---|---|---|---|---|---|
| Identifier | Infection | Skin | 1 | 2 | 3 | 4 | 6 | 7 | 9_35 | 9_36 | 9_39 | 9_56 | 9_71 | 10 | 8 | 9 | N90 | N37 | N38 | N7 |
| WAN01UQ5N_at | 8 | 2 | + | + | − | − | + | + | − | + | + | + | + | − | − | + | − | − | − | + |
| WAN01UO4E_at | 8 | 2 | + | + | − | − | + | + | − | + | + | + | + | − | − | + | − | − | − | + |
| WAN01UO4B_at | 8 | 2 | + | + | − | − | + | + | − | + | + | + | + | − | − | + | − | − | − | + |
| WAN01UO4D_at | 8 | 2 | + | + | − | − | + | + | − | + | + | + | + | − | − | + | − | − | − | + |
| WAN01UEZ7_at | 8 | 2 | + | + | − | − | + | + | − | + | + | + | + | − | − | + | − | − | − | + |
| WAN01UO49_at | 8 | 2 | + | + | − | − | + | + | − | + | + | + | + | − | − | + | − | − | − | + |
| WAN01UO47_at | 8 | 2 | + | + | − | − | + | + | − | + | + | + | + | − | − | + | − | − | − | + |
| WAN01UO46_at | 8 | 2 | + | + | − | − | + | + | − | + | + | + | + | − | − | + | − | − | − | + |
| WAN01UQ5M_at | 8 | 2 | + | + | − | − | + | + | − | + | + | + | + | − | − | + | − | − | − | + |
| WAN01UQ5L_at | 8 | 1 | + | + | − | − | + | + | − | + | + | + | + | − | − | − | − | − | − | + |
| WAN01UOJ8_at | 10 | 2 | + | + | + | + | + | + | + | + | + | + | − | − | − | − | + | − | − | + |
| WAN01UO3O_at | 10 | 2 | + | + | + | + | + | + | + | + | + | + | − | − | − | − | + | − | − | + |
| WAN01UR6F_at | 10 | 2 | + | + | + | + | + | + | + | + | + | + | − | − | − | − | + | − | − | + |
| WAN01UNYQ_at | 8 | 2 | + | + | + | + | + | + | M | M | + | M | + | + | + | M | M | M | M | M |
| WAN01UR7P_at | 10 | 2 | + | + | + | + | + | + | + | M | + | + | + | + | − | M | M | M | + | M |
| WAN01UQGI_at | 9 | 2 | + | + | + | + | + | + | M | M | + | + | + | M | + | M | M | M | + | M |
| WAN01UDU8_at | 9 | 1 | + | + | + | + | + | + | M | + | + | + | − | − | − | − | − | M | + | M |
| WAN01UEUM_at | 8 | 2 | + | + | − | − | + | + | − | + | + | + | + | − | + | − | − | − | − | + |
| WAN01UGWF_at | 10 | 2 | + | + | + | + | + | + | + | + | + | + | − | − | + | − | − | − | − | + |
| WAN01UOX6_at | 8 | 1 | + | + | − | − | + | + | − | + | + | + | + | − | − | − | − | − | − | + |
| WAN01UQ6Q_at | 10 | 2 | + | + | + | + | + | + | + | + | + | + | − | − | + | − | − | − | − | + |
| WAN01UQM5_at | 10 | 2 | + | + | + | + | + | + | + | + | + | + | − | − | − | − | + | − | − | + |
| WAN01UR2S_at | 8 | 1 | + | + | − | − | + | + | − | + | + | + | + | − | − | − | − | − | − | + |
| WAN01UR39_at | 8 | 1 | + | + | − | − | + | + | − | + | + | + | + | − | − | − | − | − | − | + |
| WAN01UR3G_at | 10 | 1 | + | + | + | + | + | + | M | + | + | + | + | M | − | M | − | − | − | + |
| WAN01UR3H_at | 8 | 1 | + | + | − | − | + | + | − | + | + | + | + | − | − | − | − | − | − | + |

| Identifier | Gene | Locus | Strain | Description |
|---|---|---|---|---|
| WAN01UQ5N_at | arcC | SE0102 | S. epidermidis ATCC12228 | Putative carbamate kinase |
| WAN01UO4E_at | argF | SE0103 | S. epidermidis ATCC12228 | Ornithine carbamoyltransferase |
| WAN01UO4B_at | | | S. epidermidis ATCC12228 | Hypothetical protein |
| WAN01UO4D_at | | SE0104 | S. epidermidis ATCC12228 | Transcription regulator Crp/Fnr family protein |
| WAN01UEZ7_at | arcD | SE0105 | S. epidermidis ATCC12228 | Arginine/oirnithine antiporter |
| WAN01UO49_at | arcA | SE0106 | S. epidermidis ATCC12228 | Arginine deiminase |
| WAN01UO47_at | | | S. epidermidis ATCC12228 | Hypothetical protein |
| WAN01UO46_at | | | S. epidermidis ATCC12228 | Hypothetical protein |
| WAN01UQ5M_at | argR | SE0107 | S. epidermidis ATCC12228 | Arginine repressor |
| WAN01UQ5L_at | | SE0110 | S. epidermidis ATCC12228 | Conserved hypothetical protein |
| WAN01UOJ8_at | | | S. epidermidis ATCC12228 | Hypothetical protein |
| WAN01UO3O_at | | | S. epidermidis ATCC12228 | Hypothetical protein |
| WAN01UR6F_at | | SE0113 | S. epidemidis ATCC12228 | hypothetical protein |
| WAN01UNYQ_at | | | S. epidermidis ATCC12228 | Hypothetical protein |
| WAN01UR7P_at | | | S. epidermidis ATCC12228 | Hypothetical protein |
| WAN01UQGI_at | | SE2072 | S. epidermidis ATCC12228 | Hypothetical protein |
| WAN01UDU8_at | | | S. aureus 85/2082 | Conserved hypothetical protein, SCCmec |
| WAN01UEUM_at | | SERP2469 | S. epidermidis RP62A | Hypothetical protein, similar to alcohol dehydrogenase |
| WAN01UGWF_at | | SAC0043 | S. aureus COL | Conserved hypothetical protein |
| WAN01UOX6_at | | | S. epidermidis SR1 | Hypothetical protein |
| WAN01UQ6Q_at | | SAC0040 | S. aureus COL | Hypothetical protein |
| WAN01UQM5_at | | SERP2502 | S. epidermidis RP62A | Similar to hypothetical protein |
| WAN01UR2S_at | | | S. epidermidis ATCC14990 | Hypothetical protein |
| WAN01UR39_at | | | S. epidermidis ATCC14990 | Hypothetical protein |
| WAN01UR3G_at | | | S. epidermidis ATCC14990 | Similar to probable specificity determinant HsdS |
| WAN01UR3H_at | | | S. epidermidis ATCC14990 | Hypothetical protein |

ix). Agr Typing

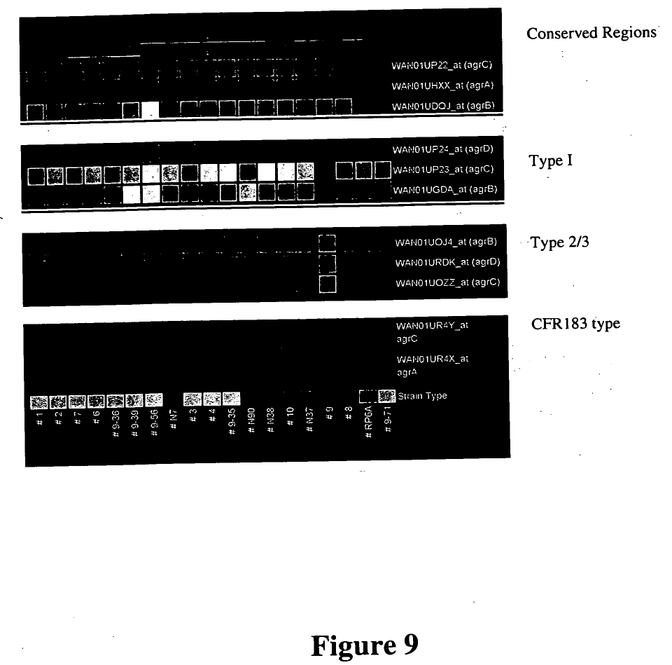

An example of the ability of the nucleic acid array of Example 1 to distinguish allelic variants is shown in FIG. 9. The agr quorum-sensing and signal transduction locus that controls the expression of many staphylococcal virulence genes is highly variable among staphylococcal species, particularly the 3′ half of agrB, the 5′ half of agrC and the gene encoding the extracellular peptide agrD. Three agr types are distinguishable on the nucleic acid array of Example 1: types 1, 2/3, and that of strain CFR 183. All strains reported here are Type 1 with the exception of strain 9, which belongs to either type 2 or type 3. The conserved regions of agrB and agrC, which are tiled separately from the variable regions, are present in all strains including strain 9 (bottom panel).

x). Discussion

This Example presents a study of the relatedness and genetic composition of 18 Staphylococcal epidermidis strains from two New York City hospitals, using the nucleic acid array of Example 1. Strains were compared by hybridization of genomic DNA on the nucleic acid array comprising 4,449 ORFs, 2,871 intergenic, and 40 tRNA and rRNA qualifiers derived from two completed S. epidermidis genome sequences and additional publicly available S. epidermidis sequences. Present/Absent calls for each strain were made based on the normalized signal intensity for each gene and several of these were confirmed by PCR analysis.

As demonstrated in U.S. patent application Ser. No. 10/859,198, nucleic acid array analysis can provide more discrimination than other methods including PFGE, ribotyping, and MLST typing. In this Example, the isolates obtained from infections are much more similar to one another than to skin isolates, and that the skin isolates are generally a more divergent group. For example, two essentially indistinguishable isolates (#1 & 2) were distinguishable from but very similar to the two isolates (#6 & 7) obtained from the same child one week later; but very different from a skin isolate (#8) obtained following treatment for the infection, one month later. One strain obtained from the skin of a health care worker (N7) was very similar to two strains obtained from infections at the same hospital. See, for example, FIG. 7.

Nucleic acid array analysis offers an unparalleled ability to determine the genetic composition of a strain, without foreknowledge of genes which may be of interest. Using a published list of known virulence factors, this Example demonstrates that all of these isolates, whether from normal skin or infection sites, carry most of these genes. By examining the 12 qualifiers derived from the several variants of the agr locus, it is showed that these strains, with one exception, are agr type I-se. All strains carry the methicillin resistance gene mecA, although the presence of other genes from SSCmec cassettes differs among the strains.

Of the more than 2700 genes in common between the infection associated- and commensal-isolates studied, 26 genes were found to be predominantly present in the infection-related strains (Table 8). Characterization of these gene products may present an opportunity to understand the molecular basis of infectivity. Of the 26 genes, many are involved in bacterial metabolism and physiology; however, this study also identified twelve proteins of unknown function, which may represent new virulence factors.

A striking difference between the infection associated strains and the commensal strains were genes that are involved in the arginine deiminase pathway (arc operon). S. epidermidis harbors two arc operons, both of which were present in the majority (73%) of the infection-associated isolates, but one was absent from most (71%) of the commensal isolates. Hence, infection may selectively enrich for staphylococci possessing a second arc operon. Consistent with this suggestion, several studies have demonstrated that arc operon transcripts are among the most abundantly produced in S. aureus and S. epidermidis biofilms. Within the closely related pathogen, S. aureus, the arc operon has been shown to be regulated by the global virulence factor regulator, RNAIII, suggesting that it is important for pathogenesis.

The arginine deiminase pathway may play a role in the formation or maintenance of biofilms on indwelling substrates. In support of this, genomic studies of staphylococcal biofilms suggest that bacteria are growing microaerobically relative to planktonic cultures. As many enzymatic reactions require oxygen, reduced oxygen availability severely limits the metabolic options available to bacteria for energy production and macromolecular synthesis. To overcome reduced oxygen availability, one would expect staphylococci growing in a biofilm to induce alternative energy generating pathways, such as the arginine deiminase pathway and the corresponding arginine transporters. Indeed, the arginine deiminase pathway is an arginine fermentation pathway that generates the small molecule phosphate-donor carbamoyl-phosphate, which is used for substrate-level phosphorylation to generate ATP.

The infection associated genes identified in this study were not previously characterized by Yao, et al., INFECT IMMUN., 73:1856-1860 (2005). In that study, an array containing a single oligonucleotide representing predicted S. epidermidis RP62A ORFs was used to compare the genomic composition of skin- and either pus- or tissue-isolates from patients with chronic prosthetic joint infections. The authors identified 39 infection-associated genes that do not overlap with the 26 genes depicted in Table 8.

### EXAMPLE 7

### Genetic Characterization of Streptococcus pyogenes Isolates

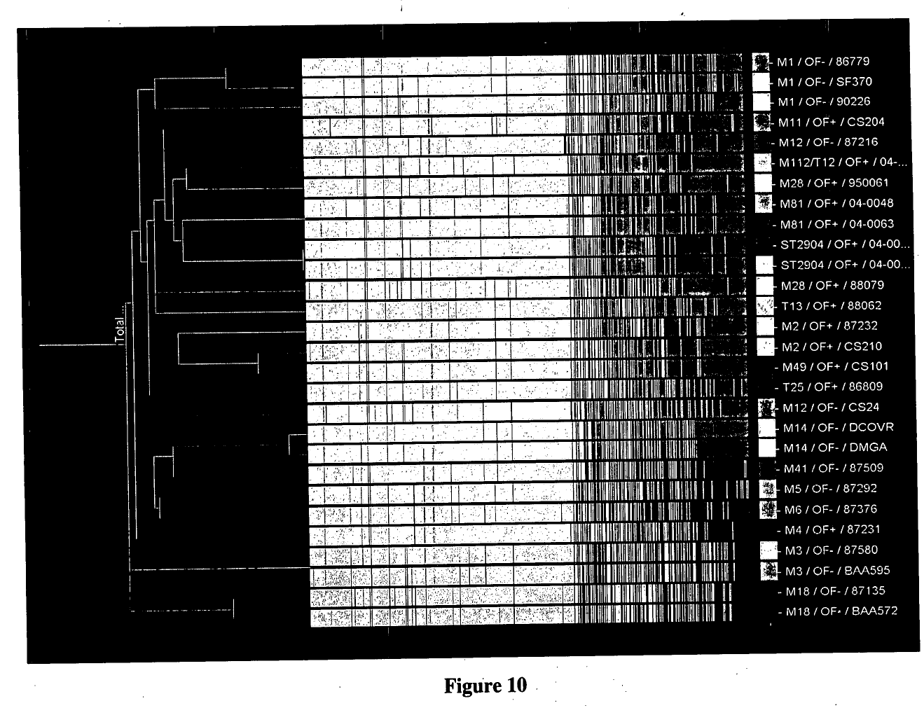

The nucleic acid array of Example 1 was used to compare the genetic composition of S. pyogenes isolates. A number of clinical isolates of S. pyogenes were clustered using normalized signal for all open reading frames on the array. FIG. 10 depicts a dendrogram showing DNA similarities among different S. pyogenes isolates. Yellow represents a gene present in the strain (positive hybridization signal), blue indicates its absence, and intermediate colors represent intermediate signals indicating a lower than average signal.

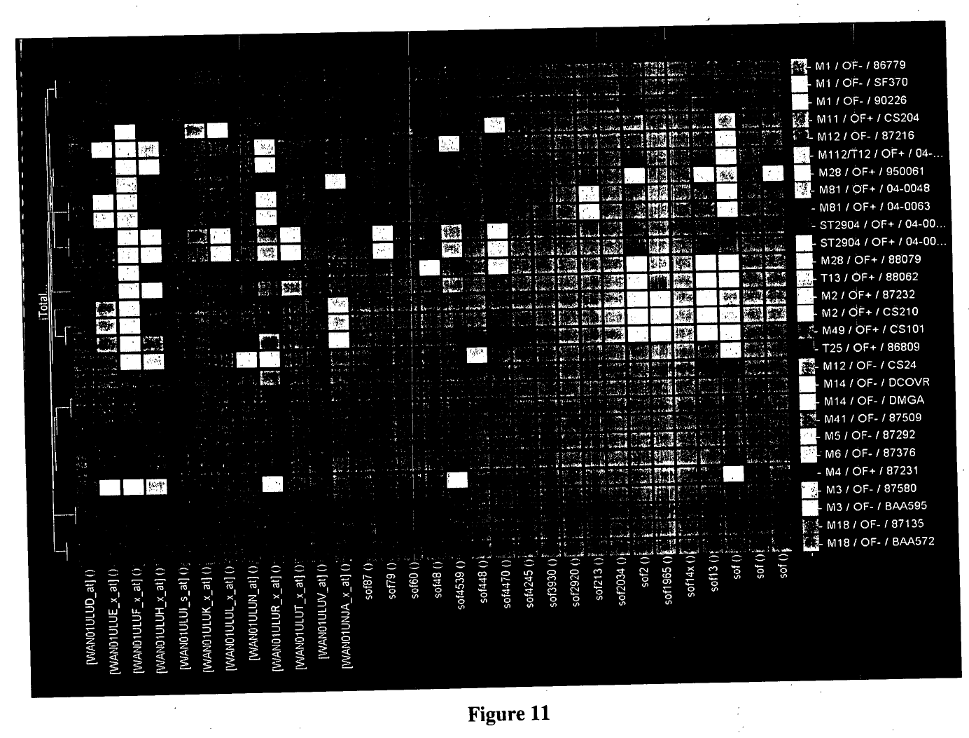

The nucleic acid array of Example 1 was also used to classify and type different S. pyogenes isolates. One of the main classifications of S. pyogenes is based on the strain's ability to turn serum cloudy. This phenotype is referred to as OF⁺ or OF⁻, and is determined by the existence or nonexistence of serum opacity factor (SOF). The sof gene is highly variable in sequence and, therefore, is represented numerous times on the nucleic acid array employed. Some qualifiers on the array represent conserved regions common to more than one gene and some represent unique regions. As shown in FIG. 11, each OF⁺ strain hybridizes to at least one sof gene on the array, while OF⁻ strains (with one exception) hybridize to none. Columns 13 to 31 in FIG. 11 represent WAN01ULUZ_x_at (sof87), WAN01ULV2_at (sof79), WAN01P979_x_at (sof60), WAN01ULV5_at (sof48), WAN01ULUS_x_at (sof4539), WAN01ULUQx_at (sof448), WAN01ULUJ_x_at (sof4470), WAN01ULUY_at (sof4245), WAN01ULUW_at (sof3930), WAN01P982_at (sof2920), WAN01ULV6_at (sof213), WAN01ULV3_s_at (sof2034), WAN01ULUO_x_at (sof2), WAN01ULUM_at (sof1965), WAN01ULUP_x_at (sof14x), WAN01UNJ9_x_at (sof13), WAN01ULUG_x_at (sof), WAN01ULUX_x_at (sof), and WAN01ULV4_at (sof), respectively. All of the qualifiers in FIG. 11 are derived from the sof gene.

The M protein genes were also used to distinguish and classify S. pyogenes isolates. The sequences of these genes are highly variable among different M types.

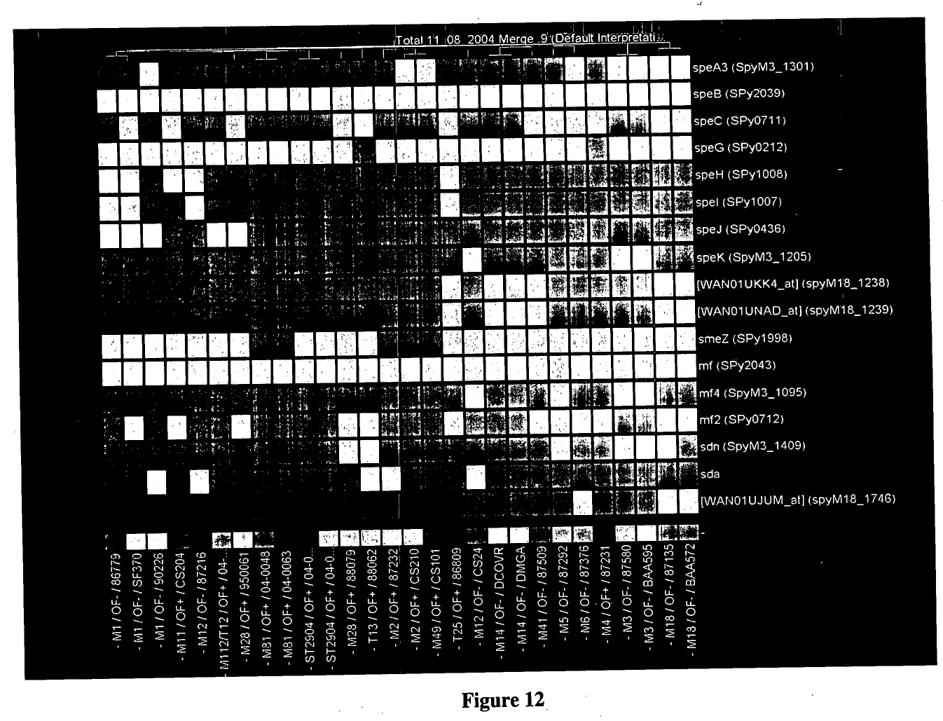

In addition, genes encoding enzymes and exotoxins were used to distinguish S. pyogenes strains. S. pyogenes secretes numerous enzymes and exotoxins. The frequency of these genes varies substantially among different S. pyogenes isolates. FIG. 12 illustrates the hybridization signals of selected enzyme and exotoxin genes in different S. pyogenes isolates. Each isolate is represented by a column and each gene by a row. Rows 1-17 represent WAN01ULSE_at, WAN01UJDW_at, WAN01UJZ_at, WAN01UJ9B_at, WAN01UKB9_at, WAN01UKU9_at, WAN01UK23_at, WAN01UNA9_at, WAN01UKK4_at, WAN01UNAD_at, WAN01UJY6_at, WAN01UMZD_at, WAN01UNEV_at, WAN01UJZR_at, WAN01UJUL_at, WAN01UJUN_at, and WAN01UJUM_at, respectively. WAN01ULSE_at, WAN01UJDW_at, WAN01UJZQ_t, WAN01UJ9B_at, WAN01UKB9_at, WAN01UKU9_at, WAN01UK23_at, WAN01UNA9_at, WAN01UKK4_at, and WAN01UNAD_at are derived from Streptococcal pyrogenic exotoxin genes; WAN01UJY6_at is derived from a mitogenic exotoxin gene; WAN01UMZD_at, WAN01UNEV_at, and WAN01UJZR_at are derived from mitogenic factor genes; and WAN01UJUL_at, WAN01UJUN_at, and WAN01UJUM_at are derived from streptodomase gene.

The nucleic acid array of Example 1 was also used for the identification of vaccine candidates for the prevention or treatment of S. pyogenes infections. Preferred vaccine candidates comprise sequences that are conserved among different S. pyogenes strains. FIG. 13 depicts exemplary qualifiers whose sequences are conversed among all of the clinical S. pyogenes isolates that were tested. Each column in FIG. 13 represents a clinical isolate and each row represents a conserved qualifier (except scpA15). Rows 1-13 represent WAN01UMWF_at (SPy0836), WAN01UKXY_at (SPy0843), WAN₀₁UNE5_at (PRSA1), WAN01UK2H_at (adcA), WAN01UKQ6_at (dppA), WAN01UMZE_at (oppA), WAN01UJHC-seg1_at (prtS), WAN01UJHC-seg2_at (prtS), WAN01UMYS_at (scpA), WAN01UMYU_at (scpA), WAN01UMYR_at (scpA), WAN01UMCN_at (scpA15), and WAN01UMSZ_at (scpB), respectively. WAN01UMWF_at and WAN01UKXY_at encode hypothetical proteins; WAN01UNE5_at, WAN01UK2H_at, WAN01UKQ6_at, and WAN01UMZE_at encode a putative protease maturation protein, a putative adhesion protein, a surface lipoprotein, and an oligopeptide permease, respectively; WAN01UJHC-seg1_at and WAN01UJHC-seg2_at encode a putative cell envelope proteinase; WAN01UMYS_at, WAN01UMYU_at, and WAN01UMYR_at encode different segments of C5A peptidase precursor; and WAN01UMCN_at and WAN01UMSZ_at encode C5A peptidase.

To further evaluate vaccine candidates, RNA was prepared from strain SF370 (M1) which had been grown to either early or late log phase, and hybridized to the nucleic acid array of Example 1. Genes that were more highly expressed in the early log phase than in the late growth phase were identified. These genes include, but are not limited to, putative ribosomal protein S1-like DNA-binding protein (25.08), C5A peptidase precursor segment (WAN01UMYS_at) (15.51), C5A peptidase precursor segment (WAN01UMYU_at) (11.16), putative amino acid ABC transporter, periplasmic amino acid-binding protein (9.26), C5A peptidase precursor segment (WAN01UMYR₁₃ at) (7.84), putative 42 kDa protein (7.21), transcription regulator—(trigger factor (prolyl isomerase)) (5.96), putative cell division protein (DivIC) (5.08), putative pantothenate kinase (4.61), streptolysin 0 precursor (3.86), 50S ribosomal protein L20 (3.59), 50S ribosomal protein L11 (3.39), collagen binding protein (2.95), putative signal peptidase 1 (2.91), putative protease maturation protein (SPy1390) (2.81), putative ABC transporter (lipoprotein) (2.63), putative cyclophilin-type protein (2.29), penicillin-binding protein (D-alanyl-D-alanine carboxypeptidase) (2.26), putative penicillin-binding protein 1b (2.17), C5a-peptidase, scpA15, SPy0843 (WAN01UKXY_at), and dppA. The number in each parenthesis indicates the fold change between the two time points.

Genes that were more highly expressed in the late log phase than in the early growth phase were also identified. Non-limiting examples of these genes include pyrogenic exotoxin B (231.9). putative ornithine transcarbamylase (78.8), streptococcal antitumor protein (possible arginine deiminase) (46.09), putative pullulanase (9.7), SPy0836 (WAN01UMWF_at) (7.07), putative maltose/maltodextrin-binding protein (5.9), putative pyruvate formate-lyase (5.16), putative ATP-binding cassette transporter-like protein (2.79), heat shock protein—cochaperonin (2.61), heat shock protein (chaperonin) (2.52), putative cell envelope proteinase (prtS) (2.13), and putative adhesion protein (adcA) (2.04).

Genes that encode conserved bacterial surface antigens can be selected by mass spectrometry or other suitable means. The expression products of these genes can be used to prepare immunogenic compositions for eliciting immune reactions against S. pyogenes.

The foregoing description of the present invention provides illustration and description, but is not intended to be exhaustive or to limit the invention to the precise one disclosed. Modifications and variations consistent with the above teachings may be acquired from practice of the invention. Thus, it is noted that the scope of the invention is defined by the claims and their equivalents.

What is claimed is:

1. A nucleic acid array comprising:
a first group of polynucleotide probes, each of which is specific to a different respective strain selected from a plurality of strains of a first species; and
a second group of polynucleotide probes, each of which is specific to a different respective strain selected from a plurality of strains of a second species.

2. The nucleic acid array according to claim 1, comprising:
at least one polynucleotide probe which is common to said plurality of strains of the first species; or
at least one polynucleotide probe which is common to said plurality of strains of the second species.

3. The nucleic acid array according to claim 1, wherein each said species is β-hemolytic Streptococcus species or a Staphylococcus species.

4. The nucleic acid array according to claim 1, wherein each said species is selected from the group consisting of Streptococcus pyogenes, Streptococcus agalactiae, and Staphylococcus epidermidis.

5. The nucleic acid array according to claim 4, wherein said plurality of strains of each said species comprises:
two or more Streptococcus pyogenes strains selected from the group consisting of SSI-1,2F3, Manfredo, MGAS315, MGAS8232 and SF370;
two or more Streptococcus agalactiae strains selected from the group consisting of 2603, A909 and NEM316; or
two or more Staphylococcus epidermidis strains selected from the group consisting of ATCC12228, ATCC14990, O-47, RP62A and SR1.

6. The nucleic acid array according to claim 4, comprising at least 100 polynucleotide probe sets, each of which is capable of hybridizing under stringent or nucleic acid array hybridization conditions to a different respective sequence selected from SEQ ID NOs: 1 to 18,598, or the complement thereof.

7. The nucleic acid array according to claim 4, comprising at least 1,000 polynucleotide probe sets, each of which is capable of hybridizing under stringent or nucleic acid array hybridization conditions to a different respective sequence selected from SEQ ID NOs: 1 to 18,598, or the complement thereof.

8. A nucleic acid array comprising:
a first group of polynucleotide probes, each of which is specific to a different respective strain selected from a plurality of strains of a first species;
a second group of polynucleotide probes, each of which is specific to a different respective strain selected from a plurality of strains of a second species; and
a third group of polynucleotide probes, each of which is specific to a different respective strain selected from a plurality of strains of a third species.

9. The nucleic acid array according to claim 8, wherein said plurality of strains of the first species comprises two or more Streptococcus pyogenes strains selected from the group consisting of SSI-1, 2F3, Manfredo, MGAS315, MGAS8232 and SF370; and said plurality of strains of the second species comprises two or more Streptococcus agalactiae strains selected from the group consisting of 2603, A909 and NEM316; and said plurality of strains of the third species comprises two or more Staphylococcus epidermidis strains selected from the group consisting of ATCC12228, ATCC14990, O-47, RP62A and SR1.

10. The nucleic acid array according to claim 9, comprising at least 100 polynucleotide probe sets, each of which is capable of hybridizing under stringent or nucleic acid array hybridization conditions to a different respective sequence selected from SEQ ID NOs: 1 to 18,598, or the complement thereof.

11. The nucleic acid array according to claim 9, wherein about 20% to about 40% of all perfect match probes on the nucleic acid array are capable of hybridizing under stringent or nucleic acid array hybridization conditions to mRNA transcripts of Streptococcus pyogenes, or the complements thereof; about 20% to about 40% of all perfect match probes on the nucleic acid array are capable of hybridizing under stringent or nucleic acid array hybridization conditions to mRNA transcripts of Streptococcus agalactiae, or the complements thereof, and about 30% to about 50% of all perfect match probes on the nucleic acid array are capable of hybridizing under stringent or nucleic acid array hybridization conditions to mRNA transcripts of Staphylococcus epidermidis, or the complements thereof.

12. A method for detecting, monitoring, classifying, typing, or quantitating a pathogen in a sample of interest, said method comprising:
hybridizing nucleic acid molecules prepared from said sample to a nucleic acid array of claim 1; and
detecting hybridization signals that are indicative of the presence or absence, gene expression, classification, typing, or quantity of said pathogen in said sample.

13. The method according to claim 12, wherein said pathogen is a β-hemolytic Streptococcus species or a Staphylococcus species.

14. A method for determining or validating antigen expression of a pathogen of interest, comprising:
hybridizing a nucleic acid sample prepared from said pathogen to a nucleic acid array of claim 1; and
detecting hybridization signals that are indicative of antigen expression in said pathogen.

15. A method of preparing or selecting an antigen for inclusion in an immunogenic composition against a pathogen of interest, comprising:
hybridizing a nucleic acid sample prepared from said pathogen to a nucleic acid array of claim 1;
detecting expression of a gene which encodes an immunogen of said pathogen; and
preparing or selecting an antigen for inclusion in an immunogenic composition that is capable of eliciting an immunogenic response against said immunogen.

16. A method of screening for agents capable of modulating gene expression in a pathogen of interest, comprising:
contacting an agent with said pathogen;
preparing a nucleic acid sample from said pathogen after said contacting; and
hybridizing the nucleic acid sample to a nucleic acid array of claim 1 to detect hybridizing signals,
wherein said hybridization signals, as compared to control signals, are indicative of whether said agent is capable of modulating gene expression in said pathogen.

17. An agent identified by the method of claim 16, wherein said pathogen is β-hemolytic Streptococcus species or a Staphylococcus species, and said agent is capable of inhibiting or reducing growth or virulence of said pathogen.

18. A polynucleotide collection comprising at least one polynucleotide capable of hybridizing under stringent or nucleic acid array hybridization conditions to a sequence selected from SEQ ID NOs: 1 to 18,598, or the complement thereof.

19. A probe array comprising:
a first plurality of probes, each of which is specific to a different respective strain of a first species; and
a second plurality of probes, each of which is specific to a different respective strain of a second species.

20. The probe array according to claim 19, wherein each said probe is an antibody capable of specifically binding to a protein product of a gene which encodes a non-intergenic sequence selected from SEQ ID NOs: 1 to 18,598.
